(19) United States
(12) Patent Application Publication
(10) Pub. No.: US 2004/0063220 A1
(43) Pub. Date: Apr. 1, 2004

(54) Substrate chemistry for protein immobilization on a rigid support

(76) Inventor: Stewart J. Lebrun, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/376,351

(22) Filed: Feb. 27, 2003

Related U.S. Application Data
(60) Provisional application No. 60/361,424, filed on Feb. 27, 2002.

(51) Int. Cl. G01N 33/543; C12M 1/34

(52) U.S. Cl. 436/518; 435/287.2

(57) ABSTRACT

A device is disclosed for immobilization of proteins which includes a hydrophobic polymeric layer, preferably PVDF, attached to a rigid support. The proteins are spotted onto the dry surface of the PVDF. In preferred embodiments, the PVDF-coated rigid support advantageously provides greater ease of handling, protein immobilization in the dry state, high spot density, low background and high dynamic range. Methods of using the disclosed microarray support are also disclosed.

### RELATED APPLICATIONS

This application claims benefit of priority under 35 U.S.C §119(e) to U.S. Provisional Application No. 60/361,424 filed on Feb. 27, 2002.

### BACKGROUND OF THE INVENTION

1. Field of the Invention

In a preferred aspect, the present invention relates to an improved substrate chemistry for protein immobilization on a rigid support.

2. Description of the Related Art

In the fields of biotechnology, bioactive species are often immobilized onto a support member to more effectively handle large numbers of the bioactive species. Indeed, array-based systems are now used extensively for DNA and RNA analysis.

Proteins are the major components of cells. They determine the shape, structure, and function of the cell. Proteins are assembled by 20 different amino acids each with a distinct chemical property. This variety allows for enormous versatility in the chemical and biological properties of different proteins. Human cells have about 100,000 genes for encoding different proteins. Despite the fact that new proteins are being discovered at an unprecedented rate, protein structure and function studies are lagging behind, mainly due to a lack of high throughput methods.

Antibodies and recombinant proteins are powerful tools for protein studies. Antibodies are a large family of glycoproteins that specifically bind antigens. A protein can be identified by its specific antibodies in immunochemical methods such as Western blot, immunoprecipitation, and enzyme linked immunoassay. Monoclonal and polyclonal antibodies against many known proteins have been generated and are widely used in both research and therapy. Genes can be readily expressed in organisms like bacteria and yeast and this has made recombinant proteins convenient and indispensable tools in protein structure and function studies. There is a growing demand for recombinant proteins, especially in large scale screening of drug targets and in clinical medicine. Today, numerous antibodies and recombinant proteins have been produced. One important issue is how to analyze proteins in large scale by using a large number of antibodies or recombinant proteins in a single experiment.

Monitoring the expressions and properties of a large number of proteins is desired in many important applications. One such application is to reveal protein expression profiles. A cell can express a large number of different proteins and these expression patterns (the number of proteins expressed and the expression levels) vary in different cell types. This difference is the primary reason that different cells have different functions. Since many diseases are caused by a change in protein expression, comparing protein expression patterns between normal and disease conditions may reveal proteins whose changes are critical in causing the disease and thus identify appropriate therapeutic targets. Methods of detecting protein expression profiles will also have other important applications including tissue typing, forensic identification, and clinical diagnosis. Protein expression patterns can be examined using antibodies in radioimmuno or enzyme-linked immunoassays, however, such methods are poorly suited for large scale analyses. Therefore, a major obstacle in profiling protein expression pattern is a lack of large scale protein screening methods.

An understanding of protein-protein interactions is important to learning how endogenous proteins carry out their function(s). Further, an understanding of protein-protein interactions is also key to the design of protein agonists and antagonists. Currently, there are several methods to detect protein-protein interactions. Co-immunoprecipitation (Harlow and Lane, 1988, Antibodies, a laboratory manual. Cold Spring Harbor Laboratory), yeast two-hybrid screening (Fields and Song, 1989, Nature, 340:245-246) and phage display library screening (Smith, 1985, Science 228:1315-1317) are among the more commonly used methods for detecting protein-protein interactions. However, there are severe limitations in these methods. In co-immunoprecipitation, a protein of interest can be precipitated with its antibody which is immobilized on agarose beads. Any other protein(s) that co-immunoprecipitated with the protein of interest can be identified by either blotting with its antibody when it is known or purification and sequencing when it is a novel protein. However, this method can not be applied to large scale identification of protein-protein interactions. In yeast two-hybrid screening, although a single yeast two-hybrid screening assay can detect many interacting proteins, it is time-consuming and prone to false positive results. Moreover, many protein-protein interactions only occur in the presence of additional cellular factors or after post-translational modifications, which may not be present in yeast. Therefore, yeast two-hybrid screening may fail to identify many important protein-protein interactions that only take place in mammalian cells. Phage display screening of protein-protein interaction suffers similar limitations.

For some applications, it may be desirable to immobilize proteins on a solid support to facilitate subsequent handling and analysis. In Western blot analysis, for example, proteins of interest are first separated by electrophoresis and then transferred onto a wetted membrane adapted to non-covalently bind the protein, such as nitrocellulose or a polyvinylidene difluoride (PVDF) membranes. The membrane-bound protein of interest can then be selected by some unique property, i.e., interaction with an antibody specific for the protein. Methods of using membranes, analogous to those used in Western blotting techniques, for protein arrays have been suggested (see e.g., U.S. Pat. Nos. 4,880,750 and 5,270,167 to Francouer, WO 97/29206 to Unger, and U.S. Pat. No. 6,197,599 to Chin et al.; the disclosures of which are incorporated herein in their entirety by reference thereto). In other applications, such as immunoprecipitation and affinity purification, agents (e.g., antibodies, ligands) are covalently conjugated onto solid supports (e.g., agarose beads) through their primary amines, sulfhydryls or other reactive groups. In general, proteins retain their abilities of interacting with other proteins or ligands after immobilization.

After the completion of the Human Genome Project there was an increased focus in protein research using new tools in the form of DNA/RNA microarrays which allowed researchers to study large quantities of genetic material. It was thought that these new tools could be applied to protein studies. However, the transition from DNA/RNA microarrays to protein microarrays brought with it new challenges because of the more complex nature of proteins versus DNA/RNA. Attempts to use existing DNA/RNA substrate surfaces for protein studies have proven to be far from satisfactory. Problems of existing surfaces include, but are not limited to, low binding affinity, the need for wet storage, diffusion of proteins across the membrane on hydrophilic surfaces, high background levels and the need to use expensive fluorescence scanners. Rather than trying to optimize these existing DNA/RNA substrate surfaces for protein studies, a new approach was pursued.

For example, Salinaro, et al (WO 01/61042 A2) broadly discloses a composite membrane which is a porous polymer layer disposed on a support. The polymer layer is generally a hydrophilic polymer, a polar polymer or a charged polymer, having an affinity for aqueous solutions. The membrane may be used for microarrays for a range of biomolecules including DNA, cDNA, RNA, mRNA, as well as at least one of oligodeoxyribonucleotides, oligoribonucleotides, antigens, proteins, peptides, lipids, lipoproteins, and polysaccharides.

Chin et al. (U.S. Pat. No. 6,197,599) disclose the use of a PVDF membrane for a protein array. However, the array of Chin, et al. does not provide for the ease of handling which is possible with the protein arrays described in the present invention.

Thus, the present state-of-the-art fails to provide a substrate for immobilizing a protein on a support, together with detection chemistries and imaging methods, which exhibits one or more of the following characteristics desirable for a practicable protein microarray: protein immobilization in a dry state; rigid support for improved handling and template-based multiple array screening; high spot density (minimal protein diffusion within the substrate during spotting); high signal to noise ratio (low background); and high dynamic range. Thus, there is a need for an improved support for a protein microarray that meets at least some of the above-mentioned desirable features.

### SUMMARY OF THE INVENTION

In one embodiment of the present invention, a substrate for protein immobilization is disclosed. The substrate comprises a rigid support and a hydrophobic polymeric layer attached to the rigid support. The hydrophobic polymeric layer has a surface chemistry adapted to immobilize a protein sample and the substrate is configured to allow immobilization of a plurality of protein samples on discrete addressable spots thereon.

In one preferred variation to the substrate, the hydrophobic polymeric layer is a PVDF layer. Preferably, the PVDF layer has a thickness of greater than about 100 μm, more preferably about 100 to 250 μm, and a high-end cutoff pore size in the range of about 0.1 to 1 μm, more preferably about 0.2 to 0.45 μm. The PVDF layer may be a sheet, a membrane, or may be formed from PVDF pellets.

In preferred embodiments of the substrate, the hydrophobic polymeric layer produces a background signal upon protein imaging using visual or fluorescent light of less than about 100 lumens. More preferably, the background signal is between about 0 and 50 lumens. Most preferably, the background signal produced by hydrophobic polymeric layer upon protein imaging is between about 0 and 15 lumens.

In one variation, the rigid support is a silanated material. Alternatively, the rigid support may be glass or plastic.

In preferred embodiments of the substrate, it further comprises a bar code. Another preferred variation to the substrate is a removable protective film. In another preferred variation, the substrate further comprises a template attached to the rigid support, wherein the template divides the hydrophobic polymeric layer into at least two distinct sections, each section being configured to allow immobilization of a plurality of protein samples, and wherein the template is adapted to allow application of different chemical reagents to the distinct sections of the polymeric layer.

In one aspect, the present invention relates to a method of preparing a protein array. The method comprises the step of spotting a plurality of protein samples at discrete, addressable locations onto a PVDF layer of a substrate, wherein the PVDF layer is in a dry state.

In another aspect, the present invention relates to a method of making a PVDF-coated rigid substrate adapted to immobilize a plurality of bioactive molecules. The method comprises applying an adhesive layer to a rigid support, adhering a PVDF membrane sheet to the adhesive layer, drying, and cutting excess PVDF membrane sheet away from the rigid support, to form the PVDF-coated rigid substrate. The adhesive is preferably selected from the group consisting of a double-sided inert adhesive microfilm, a silicone, a glue, a double-sided tape and direct chemical bonding. The rigid support may be glass or plastic. Another step in the method of making a PVDF-coated rigid substrate includes attaching a template to the PVDF-coated rigid substrate. In preferred variations, the template provides from about 2-500 wells.

In another aspect, the present invention relates to an array-based kit for screening a patient for a plurality of autoimmune diseases. The kit comprises a substrate having a layer of PVDF attached to a rigid support. The layer of PVDF has a plurality of protein antigens related to the autoimmune diseases immobilized thereon at different known locations. The kit also includes a reagent for detecting the binding of an antibody from the patient to the protein antigens.

### BRIEF DESCRIPTION OF THE DRAWINGS

FIG. 1A shows the Z-GRIP™ substrate with protective film, the film peeled back and the open format ready for use.

FIG. 1B shows Z-GRIP™ substrate in open format.

FIG. 1C shows the Z-GRIP™ substrate with an attached template to create a sub-array.

FIG. 2 shows manufacturing specifications for Z-GRIP™ arrays.

FIG. 3A demonstrates spot reproducibility when various buffer formulations are used. FIG. 3B shows membrane loading characteristics of PVDF. FIGS. 3C, 3D, 3E, and 3F show immunochemical interactions.

FIG. 4 shows background and specificity of the PVDF substrate.

FIG. 5A shows examples of multiple binding interactions on Z-GRIP™.

FIG. 5B shows a graphic of Ag—Ab binding for disease identification.

FIG. 6A shows data from longitudinal study of antigen stability on Z-GRIP™ substrate.

FIG. 6B shows additional data from longitudinal study of antigen stability on Z-GRIP™ substrate.

FIG. 7 shows titer measurements of pooled SLE patient sera.

FIG. 8 shows titer measurements of pooled SS patient sera.

FIG. 9 shows (p53) Capture Assay.

FIGS. 10A, 10B, 10C, 10D, 10E, 10F show detection of IgE on Z-GRIP™ substrate.

FIG. 11 shows standard curves for IgE.

FIG. 12 shows an example of qualitative and quantifiable data.

FIG. 13A shows a graphic of the hydrophobic nature of the Z-GRIP™ substrate.

FIG. 13B shows a graphic of chemical attachment of proteins.

### DETAILED DESCRIPTION OF THE PREFERRED EMBODIMENT

Most traditional protein studies have involved wet chemistries and porous membranes such as the polymer polyvinylidene fluoride (PVDF), which is widely used in techniques such as western blot. Experiments were carried out to evaluate if proteins could be immobilized on the surface of a membrane such as PVDF when it is in a dry state.

PVDF was adhered to a glass support using an inert double-sided adhesive microfilm. Proteins (more specifically antigens) were spotted onto the dry surface of the PVDF and after drying were allowed to interact with other proteins (more specifically antibodies) with a conjugated secondary antibody. Results obtained and presented herein demonstrate that the laminated substrate has proven to overcome all of the aforementioned problems encountered with existing substrates. In addition, the opaque nature of the membrane together with the chemical detection system allows the interactions to be detected and analyzed on a low-cost flatbed scanner using light in the visual wavelength spectrum.

In one embodiment, the present invention provides a protein microarray substrate that can be used in a dry state to immobilize proteins. A hydrophobic membrane is included that immobilizes proteins in a reduced surface area with minimal diffusion across the membrane. The laminated membrane adheres to a glass surface with a double-sided inert adhesive microfilm, and preferably includes a protective polymer layer over the PVDF substrate surface.

In one embodiment, the present invention can be used with multiple conjugated secondary antibodies such as Alkaline Phosphatase (AP), Biotin Protein A, or enzyme labels such as HRP or fluorescent dyes etc. In one preferred embodiment, the present invention optionally includes a barcode for test and/or sample identification and data archiving.

In one embodiment, the present invention provides a protein microarray with very little background noise. More specifically, the background noise for the Z-GRIP™ PVDF-coated glass slide using the alkaline phosphatase (AP) reaction for detection of proteins, visualized using a conventional flatbed scanner, is less than about 100 lumens. More preferably, the background on the Z-GRIP™ developed as above is between about 50 and 0 lumens. Most preferably, the background is from about 15 to 0 lumens. Similarly little to no background is seen when a fluorescent dye is used for protein detection on the Z-GRIP™ PVDF-coated glass slide and imaged using a fluorescent scanner. In contrast, typical backgrounds seen using commercial protein substrates, e.g., slides with epoxy surface chemistries, are above 200 lumens and usually in the 300 to 400 lumen range.

Maximum signal intensities for the Z-GRIP™ PVDF-coated glass slide in accordance with a preferred embodiment of the present invention using the Alkaline phosphatase reaction for detecting proteins and a conventional flatbed scanner for quantifying spot densities (otherwise referred to herein as “protein imaging”), analyzed using commercial imaging software (e.g., Adobe PHOTOSHOP®) are about 15,000 to 25,000 lumens. Maximum signal intensities for the Z-GRIP™ substrate using fluorescent detection chemistries and a fluorescent scanner are usually about 25,000 lumens. Although epoxy substrates also produce maximum signal intensities of about 25,000 lumens with either AP or fluorescent detection, because of the relatively high background levels seen with epoxy slides, the Z-GRIP™ PVDF-coated slides have approximately 10-fold greater total dynamic range and signal-to-noise ratios than other protein substrates. Moreover, in accordance with a preferred embodiment of the present invention, background for any detection chemistry on a PVDF-coated rigid support is less than about 1% of the maximal signal intensity, and more preferably, in the range of about 0.1% to about 1%, and most preferrably about 0.1% (e.g., 25 lumens background/25,000 lumens max signal).

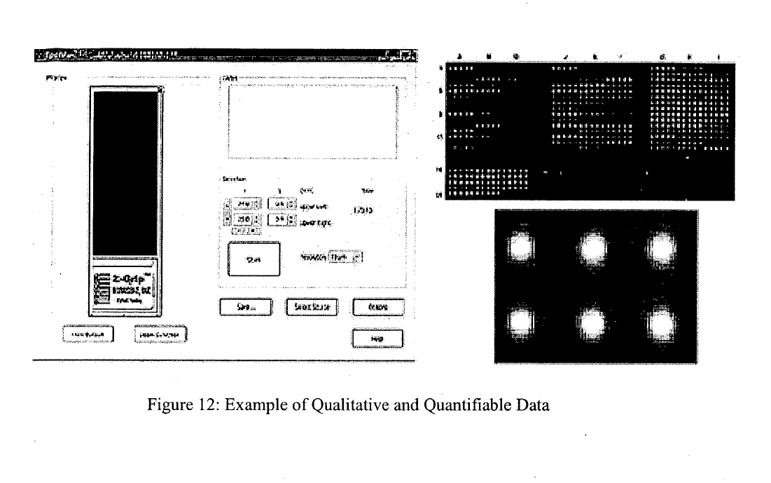

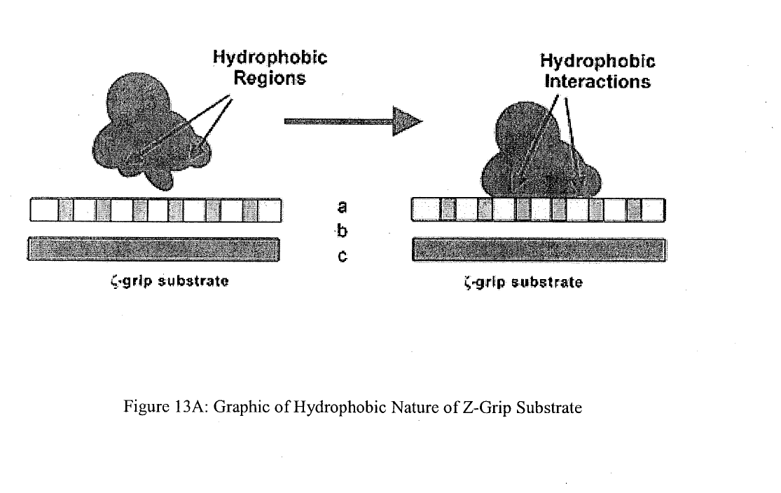

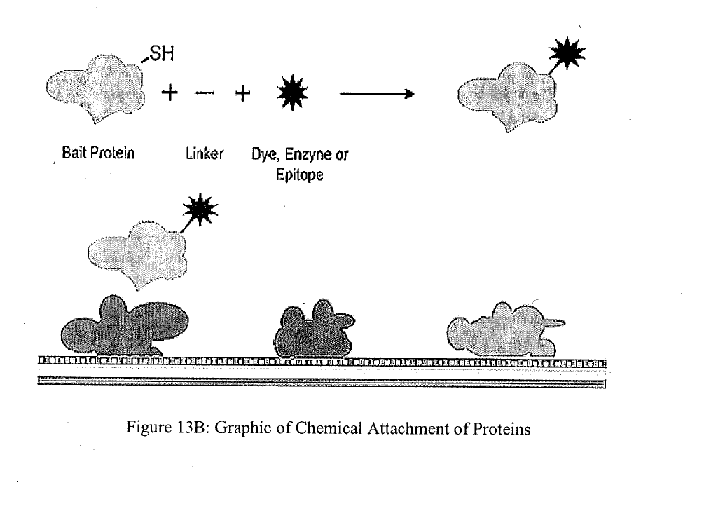

In addition to the advantages discussed above with regard to the higher signal-to-noise ratio seen with a preferred embodiment of the present invention, the Z-GRIP™ PVDF-coated rigid supports also generate enhanced assay sensitivity because the hydrophobic PVDF surface facilitates superior protein spotting/density than the hydrophilic surface chemistries typically used for protein arrays (See e.g., Salinaro et al. WO 01/61042 which teaches the criticality of using a hydrophilic surface for biomolecular arrays). As a result of the hydrophobic nature of PVDF, protein samples spotted onto the PVDF surface tend to stay in high density, very discrete micro-spots (See magnified spots shown in FIG. 12), which do not spread and diffuse through the polymeric substrate. Thus, the protein density is relatively high compared to proteins spotted onto hydrophilic substrates. As a result of the high density, the concentration of protein does not become limiting on the subsequent detection reactions (e.g., labeled secondary antibody binding). Where protein spots have spread in hydrophilic substrates, the relative protein concentrations are much lower and become limiting on the detection reactions. Consequently, the sensitivity seen using the Z-GRIP™ hydrophobic surface chemistry was observed to be approximately 1000-fold greater than sensitivities obtained with the same proteins and detection reactions on a hydrophilic surface.

In one embodiment, the present invention provides a protein microarray with the capacity to immobilize up to 20,000 proteins in the open array format.

The term “immobilize,” and its derivatives, as used herein refers to the attachment of a bioactive species directly to a support member or to a support member through at least one intermediate component. As used herein, the term “attach” and its derivatives refer to adsorption, such as, physisorption or chemisorption, ligand/receptor interaction, covalent bonding, hydrogen bonding, or ionic bonding of a polymeric substance or a bioactive species to a support member. Although the substrate chemistries of the present invention are adapted to immobilize any proteins, peptides, or polypeptides, in some embodiments of the invention, protein antigens are disclosed as being immobilized. Accordingly, the terms “antigens” and “proteins” are used interchangeably throughout the disclosure unless explicitly otherwise indicated.

Related methods of immobilizing bioactive molecules, in particular, nucleic acids, on polymeric substrates are disclosed in U.S. Pat. No. 5,897,955 to Drumheller and U.S. Pat. No. 6,037,124 to Matson; the disclosures of which are incorporated herein in their entirety by reference thereto.

This work resulted from our attempts to perform immunochemistry using antigens printed by a commercial DNA/RNA/Protein printer. The present inventors found that commercially available substrates and chemistries developed for nucleotides are not optimal for protein binding or immunochemistries. Various derivitized slides including aldehyde, epoxide, amine, L-lysine were not adequate for our requirements. Our suspicion is that binding chemistries utilized to linearize nucleotides for hybridization are not optimal for protein-protein or protein-antibody interactions. It is likely that aggressive binding of these substrates destroys secondary and tertiary protein structures and to the extent these structures are altered, epitopes vital for immuno or protein-protein assays are altered.

PVDF membrane is often used for the western blotting technique. This method involves a pre-soaking step of membrane in methanol to solubilize and the addition of methanol to buffers. The membrane must be kept in the methanol buffer or proteins will not transfer to membrane. This is often the case when there are large areas on a membrane where there was no transfer due to a bubble. In addition to being hydrophobic, PVDF membrane is hard to handle and will not lie flat during printing. These physical and chemical limitations make PVDF membrane an inappropriate and poorly suited surface for protein arrays.

We have developed a method to utilize PVDF membrane, sheets or pellets for immunochemistry and protein-protein interaction studies. Two modifications include: (1) adhering PVDF to a rigid support using an inert double-sided adhesive film or silicone, epoxy or other glues, double sided tape or direct chemical bonding to silanated slides, and (2) a printing buffer that both protects protein three-dimensional integrity and allows adherence to PVDF under dry printing conditions without membrane soaking in solvent (e.g., methanol) and associated diffusion.

Materials:

Protein-immobilizing polymer: commercially available PVDF sheets or membranes. PVDF pellets may also be used in some modes of the invention.

Solid substrate: glass slides, plastic or other flat surfaced material. Optionally, the solid substrate may be silanated.

Adhesion material: Adhesive materials include commercially available double-sided adhesive film, silicon sealant, epoxy or other glue or suitable double sided tape, and direct chemical bonding.

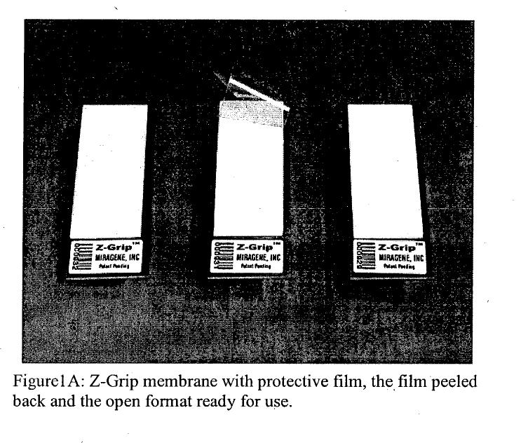

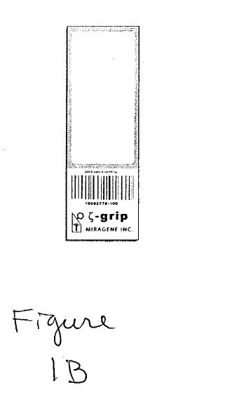

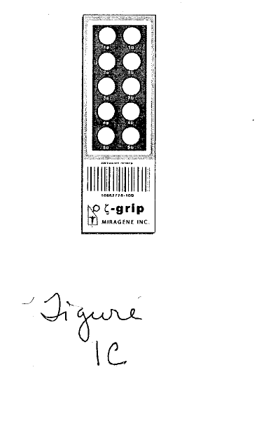

In addition to an open format shown in FIG. 1B, another preferred embodiment of the present invention can be used with an attached template to provide multiple wells or sub arrays so that separate chemistries can be performed on the same slide.

The number of individual wells could be 2 to several hundred. FIG. 1C shows a sub-array with 10 wells, each capable of 1,000 interactions.

One application is the separation of replicate arrays from each other on the same slide to allow patient comparisons or titrations. One or more steps can be performed in the small well then washing and other steps can be performed with larger volumes of solution across the whole slide.

In one embodiment, the present invention provides a three dimensional porous membrane attached to a solid support such as glass with an inert polymer. The three dimensional substrate captures and protects proteins in the porous membrane. The porous membrane has a thickness of greater than about 100 μm, more preferably approximately 100-500 μm, and most preferably between about 100-250 μm. The pore size is any pore size conventionally used for biological materials, particularly peptides and polypeptides. The high-end cutoff pore size is preferably between about 0.1 and 1 μm. More preferably, a pore size of 0.2 or 0.45 μm is used and most preferably a pore size of about 0.45 μm. Note that these pore sizes refer to maximum pore size and that there may be a range of smaller pores, below the cutoff value, present on the membrane. These characteristics help maintain the morphology of the proteins. Proteins spotted onto the substrate surface maintain their integrity, providing increased sensitivity and assay consistency.

In one embodiment, the present invention is an effective tool for studying protein-antibody, antibody-protein, protein-protein and protein-drug interactions.

In one embodiment, the array substrate (Z-GRIP™) is assembled by hand on the laboratory bench. Under clean conditions the protective coating on one side of an inert double-side adhesive film is removed and attached to a solid support such as a glass slide. A sheet of PVDF is placed on the laboratory bench face down with the protective cover still in place. The remaining protective cover on the adhesive film is removed and the solid support is then pressed firmly onto the sheet of PVDF and allowed to dry. Using a sharp instrument, e.g., a razor blade, exacto knife etc., the PVDF membrane is trimmed to the size of the solid support. As an alternative to an inert double-sided adhesive film, other adhesive materials such as silicone, glue or double-sided tape can be used.

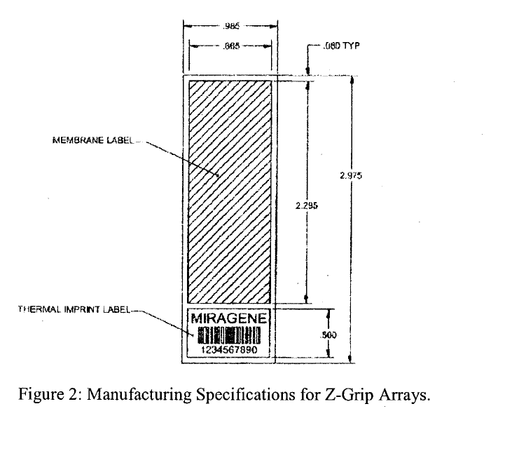

In a preferred embodiment shown in FIG. 1A the Z-GRIP™ protein array is manufactured automatically under clean conditions. A large roll (approximately 1100 inches in length and 11 inches wide) of PVDF (obtained from Millipore Corporation) mounted on a 3.25-inch core is attached to a cutting and lamination machine. The machine automatically laminates a protective film to the upper side of the PVDF and an inert double-sided adhesive film with extended liner to the backside and cuts the sheets into the preferred size depicted in FIG. 2 for automatic placement on 3″×1″ glass slides.

In another embodiment of the present invention, a layer of PVDF may be formed on a solid support by melting the polymer and applying it to the solid support. Modification of the PVDF chemistry is also deemed to fall within the scope of the present invention. Modifications may include carboxylation, amidization, and introduction of other reactive groups to the PVDF in order to promote immobilization of different bioactive species. In one embodiment, solid PVDF supports may be prepared by molding of the melted polymer.

Assays on Microarray Supports

As a result of many years of scientific research into autoimmune disease, the present inventors observed a pattern of bands in a Western blot assay which was unique for each individual. These bands represented a novel class of autoantibodies, which are not only unique but remain constant for decades and maybe for life. Further investigation of this phenomenon resulted in the development of a simple and reliable method (antibody profile assay, “Antibody Fingerprinting”) of identifying one individual from the next, as disclosed in U.S. Pat. Nos. 4,880,750 and 5,270,167 to Francoeur; the disclosures of which are incorporated herein in their entirety by reference thereto.

While the “Antibody Fingerprinting” method offers an exciting opportunity with multiple applications in the various fields of identification, the potential of utilizing a patient's autoantibody profile to provide an early warning of the onset of autoimmune disease was another aspect of Francoeur's work. However, at the time the Francoeur assay was formatted, it did not lend itself to a specific diagnosis of the many closely related and often overlapping disease syndromes that display autoantibodies. Further, it did not address some technical problems associated with protein spotting and analysis on membrane strips. Recently, new technology in the form of DNA arrays (biochips) has emerged, in which microarrays are formed on rigid supports that facilitate the application and commercialization of this work.

The present inventors have successfully demonstrated the feasibility of transferring the identification methods from a Western blot assay to an array format and have used this technology for the identification of specific antigens for detecting Systemic Lupus Erythematosus (SLE). As a result the feasibility of using disease markers in an array format to differentiate between patients with SLE and normal healthy patients had been demonstrated.

Compared to DNA chips, the challenge of successfully making protein chips is much greater because of the more complex make-up of proteins versus DNA. Whereas DNA consists of strands of only four basic chemical units (A, T, C, G), proteins are large complex molecules made up of a variety of convoluted folds of 20 amino acids. As a result the current substrate chemistries used for binding DNA fragments to an array surface are not entirely suitable for the more complex protein binding requirements. Optimization of protein substrate chemistry is a key and critical element to success in the development and commercialization of Protein Chips.

Some autoimmune disease markers used today, such as the large multi sub-unit protein SSA/Ro, are not specific to one singular disease because anti-SSA/Ro autoantibodies are present in several disease states. In this case “the overlapping symptoms” can be indicative of Systemic Lupus Erythematosus (SLE), Sjogren's Syndrome (SS) or fetal lupus with related fetal heart block syndrome (FHB). These “overlapping symptoms” occur as a result of the autoantibodies attacking different regions (epitopes) of the same large protein. One region involved in DNA binding (Leucine zipper epitope) is related to SLE symptoms, a second region (epitope) is associated to SS, while a third region (epitope) is similar in structure to fetal heart channels. FHB results from maternal antibodies crossing the blood placenta barrier (mother and fetus share the same antibodies) and attacking the fetal heart resulting in heart failure and miscarriage or stillbirth. It may be possible to separate these 3 regions of the SSA/Ro protein. If successful this could lead to a diagnostic test that can differentiate between the “overlapping symptoms” of the autoimmune diseases described above. This would be a first step towards the development of a therapy against maternal antibody related fetal heart block.

In another embodiment, the present invention is directed to a novel method for discriminating and positively identifying the source of a biological sample used for diagnostic purposes by linking the diagnostic test results to an antibody profile of the biological sample. The invention is based on the principle that humans and other animals have unique sets of antibodies. These antibodies are referred to as individual-specific (“IS”) antibodies. When IS antibodies are reacted with a random number of antigens, such as human HeLa cell antigens, certain IS antibodies specifically bind to certain antigens. IS antibody/antigen binding complexes are generally referred to as immune complexes.

In one embodiment, an antibody profile can be linked to an HIV diagnostic assay. The assay is performed in a reaction vessel, such as a PDVF-coated solid support with a plurality of wells or reaction cells. A first subset of reaction cells is used to generate an antibody fingerprint and a second subset of reaction cells used for the purposes of diagnosing HIV. The results obtained from the second subset of reaction cells is generally referred to as a diagnostic profile. With the exception of reaction cells used for negative and positive controls, in one embodiment the first subset of reaction cells are bound with antigens derived from HeLa cells and the second subset of reaction cells are bound with antigens that are specific for antibodies directed at HIV.

To each reaction cell, biological sample diluted in the appropriate buffer is added. The antigen/biological sample mixture is allowed to react for a time sufficient to permit immune complexes to form between the bound antigens and individual-specific antibodies in the biological sample, including any antibodies specific to HIV. Each reaction well is then washed to remove nonspecific binding and the immune complexes are identified. In one embodiment, the immune complexes are identified using a detector molecule such as an anti-immunoglobulin antibody or secondary antibody that has been labeled with an enzyme, a fluorophore, or a chemiluminescent substance. The secondary antibody binds to immune complexes, but not antigen alone. After a second washing, the amount of secondary antibody in each reaction cell is quantified. For example, if the secondary antibody was labeled with an enzyme, the appropriate enzyme substrate would be added and the amount of product, which is usually colored, determined spectrometrically. Finally, the spectrometric data from the antibody fingerprint and the diagnostic profile are digitized and stored in a computer. Therefore, the digitized antibody fingerprint linked diagnostic test results can be compared to previous or subsequent antibody fingerprints to ensure that the biological sample tested was from the same source or the source in question.

Components of Protein Analysis Using Microarrays (Z-GRIP™)

The discussion below is divided into five sections corresponding to five steps of one possible embodiment of the present invention which include: (1) obtaining suitable reactants; (2) obtaining a suitable reaction vessel; (3) obtaining a biological sample and reacting the biological sample with the reactants; (4) detecting and quantifying reactions, or lack of reactions, between reactants and analytes in the biological sample; and (5) digitizing the results.

Reactants

One step in practicing the method of one possible embodiment of the present invention is to obtain appropriate reactants. A reactant is defined broadly to include any molecule which can be used to measure the presence of a molecule in a biological sample referred to as an analyte. Hence reactants can be bacterial, viral, or mammalian cell antigens, hormones, drugs, receptors, tumor markers, or numerous other substances. Moreover, the reactant can be natural or synthetic, a nucleic acid or a peptide, or combinations thereof. In some embodiments, the reactant is an antigen. An antigen is defined as a molecule that is bound by an antibody.

Alternatively, reactants can be synthesized using recombinant technology well known in the art. Genes that code for many viral and bacterial proteins have been cloned and thus large quantities of highly pure proteins can be synthesized quickly and inexpensively. Likewise, the genes that code for many eukaryotic and mammalian membrane bound receptor, growth factors, cell adhesion molecules, steroids and regulatory proteins have been cloned and are useful as reactants. Many recombinant proteins such as transforming growth factor-α, acidic and basic fibroblast growth factor, interferon, insulin-like growth factors, and various interleukins from different species are commercially available from, for example, Promega Corporation, Madison, Wis.

In most instances, the entire polypeptide need not be used as a reactant. For example, any size or portion of the polypeptide that contains at least one epitope (i.e., the critical portion of the polypeptide recognized during an immune response) will suffice. In another example, the reactant may be the catalytic region or subunit of a protein which catalyzes a reaction using an analyte in the biological sample, such as the catalytic region or subunit of a protein kinase.

One skilled in the art will appreciate that the signal-to-noise ratio of the protein array or diagnostic profile will improve with the purity of the reactants used. Hence, depending on the signal-to-noise ratio desired, the reactant can be further purified, for example, by ammonium sulfate precipitation, size exclusion, dialysis or other methods well known in the art.

Peptide, polypeptide and protein antigens for use in the present invention can be purified by any of the means known in the art. See, e.g., Guide to Protein Purification, ed. Deutscher, Meth. Enzymol. 185, Academic Press, San Diego, 1990; and Scopes, Protein Purification: Principles and Practice, Springer Verlag, N.Y., 1982.

Antigen Array

An array is used in the present disclosure to mean an arrangement of molecules, particularly biological macromolecules (such as antigens, polypeptides or nucleic acids) in addressable locations on a substrate. A “microarray” is an array that is miniaturized so as to require microscopic examination for evaluation.

In preferred embodiments, the proteins are attached to rigid supports. These supports may be plates (glass or plastics), preferably coated with membranes made of PVDF, or other suitable hydrophobic polymeric material. In a preferred embodiment, as discussed above, the rigid supports are PVDF-coated supports. When interrogated with a sample, the binding of antibodies in the sample to the array (possibly producing a pattern) indicates the relative binding affinity of the antibodies for each of the immobilized polypeptides. Characteristics of binding interactions are discussed in greater detail below.

Proteins can be directly deposited at high density on a support, which in some preferred embodiments is as small as a microscopic slide. Similar technology was developed for making high density DNA microarray (Shalon et al., Genome Research, 1996, 6(7):639-645). Proteins can also be immobilized indirectly on the support. For instance, an antigen binding composition can be printed on a support. Protein antigens are then immobilized on the support through their interactions with antigen binding composition. Recombinant fusion proteins can be immobilized through the interaction between their tags and the ligands printed on the support. Regardless of the immobilization method, after agents are immobilized, the support can be treated with 5% non-fat milk or 5% bovine serum albumin (or other suitable protein blocker known in the art) for several hours in order to block non-specific protein binding.

Once reactants have been isolated or synthesized, they are added to a reaction vessel to form an array. In a preferred embodiment, the reaction vessel is a PVDF-coated rigid support. Regardless of the material or configuration of the array substrate, each location where at least one reactant is contained or bound, or could be contained or bound, may be referred to as a reaction cell or address.

With the exception of the reaction cells used as negative controls, at least one reactant is added to a plurality of reaction cells. Reactants may be in solution, bound to the reaction vessel, or bound to another surface, such as latex, polystyrene, magnetic, or glass beads, which are in turn confined or bound to the reaction vessel. In one preferred embodiment, the reaction vessel is a PVDF-coated solid support.

Within an array, each arrayed molecule is addressable, in that its location can be reliably and consistently determined within the at least two dimensions of the array surface. Thus, in ordered arrays the location of each antigen, peptide, polypeptide or partially purified lysate fraction is assigned at the time when it is spotted onto the array surface and a key may be provided in order to correlate each location with subsequent antibody binding patterns or fingerprints. Often, ordered arrays are arranged in a symmetrical grid pattern, but antigens could be arranged in other patterns (e.g., in radially distributed lines or ordered clusters). The many spots of an antigen array can be arrayed in the shape of a grid, although other array configurations can be used so long as the spots of the array are addressable.

In one preferred embodiment of the antigen array, each antigen has been spotted onto the array twice to provide internal controls. Alternatively, a greater number of replicates may be desirable in some instances. Thus, the number of replicates may range from 1 to 10, more preferably from 1 to 4 and most preferably from 1 to 2. The duplicate antigens may be positioned in a pair of horizontally adjacent addresses of the array. However, as long as the locations of the duplicate antigens in the array are known, the relative positions are not important.

Arrays may include a plurality of antigens “spotted” at assignable locations on the surface of an array substrate. In certain embodiments, polypeptides are deposited on and bound to the array surface in a substantially native configuration, such that at least a portion of the individual polypeptides within the spot are in a native configuration. Such native configuration polypeptides are capable of binding to or interacting with molecules in solution that are applied to the surface of the array in a manner that approximates natural intra- or intermolecular interactions. Thus, binding of a molecule in solution (for instance, an antibody) to an antigen immobilized on an array will be indicative of the likelihood of such interactions in the natural situation (ie., within a cell). In other embodiments of the antigen array, the peptide/polypeptides may be denatured, reduced and/or otherwise chemically pretreated (e.g., to remove sugars).

In certain arrays of the invention, one or more location/address on the array is occupied by a pooled mixture of more than one substantially pure antigens/polypeptides (e.g., chromatography fractions of a crude cell lysate or tissue extract). All of the locations on the array may contain pools of peptides, or only some of the locations. In some circumstances it may be desirable to array a polypeptide associated with one or more non-target polypeptides, for instance a stabilizing polypeptide or linker molecule. In addition, the native conformation of certain binding sites on proteins can only be assayed for antibody binding when the antigen is associated with other molecules, for instance when a polypeptide natively exists as one subunit of a multimeric complex. Pooled arrays include those on which one or more of the locations contain a multimeric polypeptide complex. In the case of such an array, it is envisioned that different antibody molecules may bind to different determinants within the complex of pooled or linked antigens.

In accordance with one embodiment of the present invention, bound antibody molecules or other detector/developer molecules (e.g., labeled aptamers) can be stripped from an array, in order to use the same array for another patient sample analysis, once the array results, optionally including an antibody fingerprint and diagnostic test are recorded and stored. Any process that will remove essentially all of the bound antibody molecules from the array, without also significantly removing the immobilized proteins/antigens of the array, can be used with the current invention. By way of example only, one method for stripping a protein array is by washing it in stripping buffer (e.g., 1 M (NH)₂SO₄, and 1 M urea), for instance at room temperature for about 30-60 minutes. Usually, the stripped array will be equilibrated in a low stringency wash buffer prior to incubation with another sample.

In microarrays, a common feature is the small size of the protein array, for example on the order of a squared centimeter or less. A squared centimeter (1 cm by 1 cm) is large enough to contain over 2,500 individual antigen spots, if each spot has a diameter of 0.1 mm and spots are separated by 0.1 mm from each other. A two-fold reduction in spot diameter and separation can allow for 10,000 such spots in the same array, and an additional halving of these dimensions would allow for 40,000 spots. Using microfabrication technologies, such as photolithography, pioneered by the computer industry, spot sizes of less than 0.01 mm are feasible, potentially providing for over a quarter of a million different target sites. The power of microarray format resides not only in the number of different antigens that can be probed simultaneously, but also in how little protein is needed for the spot. Samples applied to individual spots on a microarray will usually be less than 1 pmol in each spot, for instance, about 8 pmol, about 0.5 pmol, about 0.3 pmol, about 0.1 pmol, about 0.05 pmol or less.

In addition, the surface area of protein application for each “spot” will influence how much protein is immobilized on the array surface. Thus, a larger spot (having a greater surface area) will generally accept or require a greater amount of target molecule than a smaller sample spot (having a smaller surface area).

The antigen itself (e.g., the length of the peptide or polypeptide, its primary and secondary structure, its binding characteristics in relation to the array substrate, etc.) will influence how much of each antigen is applied to an array. Optimal amounts of antigen for application to an array of the invention can be easily determined, for instance by applying varying amounts of the antigen to an array surface and probing the array with an antibody known to interact with that antigen. In this manner, it is possible for one of ordinary skill in the art to empirically determine of range of antigen amounts that produce reproducible and interpretable results.

Another way to describe an array is its density—the number of antigens in a certain specified surface area. For microarrays, array density will usually be one target per squared centimeter or more, for instance about 50, about 100, about 200, about 300, about 400, about 500, about 1000, about 1500, about 2,500, about 5,000, about 10,000, about 50,000, about 100,000 or more targets per squared centimeter.

Antigens on the array may be made of oligopeptides, polypeptides, proteins, or fragments of these molecules. Oligopeptides, containing between about 8 and about 50 linked amino acids, can be synthesized readily by chemical methods. Photolithographic techniques allow the synthesis of hundreds of thousands of different types of oligopeptides to be separated into individual spots on a single chip, in a process referred to as in situ synthesis, as has been done with oligonucleotide arrays.

Longer polypeptides or proteins, on the other hand, contain up to several thousand amino acid residues, and are not as easily synthesized through in vitro chemical methods. Instead, polypeptides and proteins for use in antigen arrays are usually expressed using one of several well known cellular expression systems, including those described above. Alternatively, proteins can be isolated from their native environment, for instance from tissue samples or cell cultures, or from expression chambers in the case of engineered expressed polypeptides. After extraction and appropriate purification, the polypeptide can be deposited onto the array using any of a variety of techniques.

In the methods disclosed in this applications, antigens can be delivered to the substrate of the array by various different mechanisms. One is by flowing within a channel defined on predefined regions of the array substrate. Typical “flow channel” application methods for applying polypeptides to arrays are represented by dot-blot or slot-blot systems (see, e.g., U.S. Pat. Nos. 4,427,415 and 5,283,039). One alternative method for applying the antigens to the array substrate is “spotting” the antigens on predefined regions (each corresponding to an array address). In a spotting technique, the target molecules are delivered by directly depositing (rather than flowing) relatively small quantities of them in selected regions. For instance, a dispenser can move from address to address, depositing only as much antigen as necessary at each stop. Typical dispensers include an ink-jet printer or a micropipette to deliver the antigen in solution to the substrate and a robotic system to control the position of the micropipette with respect to the substrate. Quill, split quill or channel spotting pins may be used in accordance with one embodiment of the present invention to deposit or “spot” nanoliter and/or sub-nanoliter quantities of a sample protein. For example, dispensing robots such as Telechem International Inc.'s SPOTBOT™ utilize delivery technology (See e.g., U.S. Pat. No. 6,101,946, incorporated herein in its entirety by reference thereto), which is adapted for use in forming the microarrays disclosed herein. In other embodiments, the dispenser may include a series of tubes, a manifold, an array of pipettes, or the like so that the proteins can be delivered to the reaction regions simultaneously.

In a preferred embodiment, the proteins/antigens are deposited on the array substrate in such a way that they are substantially irreversibly bound to the array. For example, a target may be bound such that no more than 30% of the polypeptide on the array at the end of the binding process can be washed off using buffers (e.g., low or high salt buffers or stripping buffers). In other embodiments, no more than 25%, no more than 20%, no more than 15%, no more than 10%, no more than 5%, or no more than 3% of the antigen on the array at the end of the binding process can be washed off.

Depending on the array substrate used, the substrate alone may substantially irreversibly bind the antigen without further linking being necessary (e.g., nitrocellulose and PVDF membranes). In other instances, a linking or binding process must be performed to ensure binding of the antigens. Examples of linking processes are known to those of skill in the art, as are the substrates that require such a linking process in order to bind polypeptide molecules. The antigen polypeptides optionally may be attached to the array substrate through linker molecules.

In certain embodiments, the regions of the array surface that do not contain any antigens are blocked in order to prevent or inhibit binding of the antibody molecules directly to the array surface.

It is beneficial in certain embodiments to apply a known amount of each antigen to the array. For example, where the diagnostic test antigens are applied, it may be useful to have a known amount of the antigen. Moreover, in some modes, several doses of the known test antigens may be useful to quantitate antibody titer levels in the patient sample. In particular embodiments, an essentially equal amount of each antigen is applied to each spot. Quantification and equivalent application of the antigen permits comparison of antibody binding affinity between the different antigens. Measurements of the amount of specific proteins may be carried out through many techniques well known in the art.

Arraying pooled antigens spotted on the array is also a powerful tool in hi-throughput technologies for increasing, the information that is yielded each time the array is assayed. Methods for analyzing signals from arrays containing pooled samples have been described, for instance in U.S. Pat. No. 5,744,305, incorporated herein by reference in its entirety.

Biological Sample

The biological sample can be from various bodily fluids and solids, including blood, saliva, semen, serum, plasma, urine, amniotic, pleural or cerebrospinal fluid. Depending on the detection method used, it may be required to manipulate the biological sample to attain optimal reaction conditions. For example, the ion concentration of the biological sample may be adjusted for optimal immune complex formation, enzymatic catalysis, DNA hybridization, or DNA synthesis. It is also contemplated that the portion of the biological sample used to generate an antibody fingerprint can be treated differently than the portion of the biological sample used to generate the diagnostic profile. For example, a portion of the biological sample could be adjusted for optimal immune complex formation and another portion of the sample could be adjusted for optimal PCR condition.

Once the biological sample is optimized for reaction conditions, a small portion of the sample is added to each reaction cell of the vessel. The amount of biological sample required per reaction cell will depend on the biological assay performed and the sensitivity of the detection method used. Analytes in the biological sample will interact with the reactants in the reaction vessel.

Binding Interaction

The binding interaction between the antibodies associated with a patient's clinical state, e.g., antibodies against viruses, bacteria and other pathogens, and the antigens spotted on the array focuses on the association between two substances or molecules. The antigen arrays are used to detect binding of an antibody to one or more antigens of the array. Antibody is considered to “bind” to an antigen of the array if, after incubation of the antibody (usually in solution or suspension) with or on the array for a period of time (usually 5 minutes or more, for instance 10 minutes, 20 minutes, 30 minutes, 60 minutes, 90 minutes, 120 minutes or more), a detectable amount of the antibody associates with an antigen of the array to such an extent that it is not removed by being washed with a relatively low stringency buffer (e.g., 100 mM KCI). In some applications, e.g., where nonspecific binding is elevated, higher stringency buffers may be used.

Washing can be carried out, for instance, at room temperature, but other temperatures (either higher or lower) can also be used. Antibodies will bind different antigens to different extents, and the term “bind” encompasses both relatively weak and relatively strong interactions. Thus, some binding will persist after the array is washed in a higher salt buffer (e.g., 500 mM or 1000 mM KCI).

The relative intensity of the binding signals from individual antigen or polypeptide spots is indicative of the relative affinities of the antibodies for those antigen molecules (assuming that the same number of antibody binding sites are immobilized at each address on the array). Quantification of the binding pattern of an array-sample combination can be carried out using any of several existing techniques, including scanning the signals into a computer for calculation of relative density of each spot. Quantitation methodology is discussed in greater detail below.

Detection and Quantification of Reactant/Analyte Interactions

The prior art is replete with methods for detecting reactions and interactions between two molecules. One possible embodiment of the present invention may be modified by one skilled in the art to accommodate the various detection methods known in the art. The exact detection method chosen by one in the art will depend on several factors, including the amount of biological sample available, the biological sample type, the stability of the biological sample, the stability of the reactant and the affinity between the reactant and analyte. Moreover, as discussed above, depending on the detection methods chosen, it may be required to modify the reactant and biological sample.

While these techniques are well known in the art, examples of a few of the detection methods which could be utilized to practice one possible embodiment of the present invention are briefly described below.

Immunoassays

There are many types of immunoassays known in the art. The most common type of immunoassay are competitive and non-competitive heterogeneous assays such as enzyme-linked immunosorbent assays (ELISA). In immunoassays the reactant is an antigen. In a noncompetitive ELISA, unlabeled antigen is bound to a solid phase such as a PVDF-coated solid support. Biological sample is combined with antigens bound to the reaction vessel and antibodies (primary antibodies) in the biological sample are allowed to bind to the antigens forming immune complexes. After immune complexes have formed, excess biological sample is removed and the support is washed to remove nonspecifically bound antibodies. Immune complexes are then reacted with an appropriate enzyme-labeled anti-immunoglobulin (secondary antibody). Anti-immunoglobulins recognize bound antibodies, but not antigens. Anti-immunoglobulins specific for antibodies of different species, including human, are well known in the art and commercially available from Sigma Chemical Company, St. Louis, Mo. and Santa Cruz Biotechnology, Santa Cruz, Calif. After a second wash step, the enzyme substrate is added. The enzyme linked to the secondary antibody catalyzes a reaction which converts substrate into product. When excess antigen is present, the amount of catalyzed product is directly proportional to the amount of antigen specific antibodies (analyte) in the biological sample. Typically, the reaction product is colored and thus measured spectrophotometrically using UVNIS technology and equipment well known in the art.

Sandwich or capture assays can also be used to identify and quantify immune complexes. Sandwich assays are a mirror image of non-competitive ELISAs, antibodies are bound to the solid phase and antigen in the blood is measured (analyte). These assays are particularly useful in detecting antigens that are present at low concentrations having multiple epitopes. This technique requires excess antibody to be attached to a solid phase, such as the reaction vessel or magnetic beads. The bound antibody is then incubated with the biological sample and antigens in the biological sample are allowed to form immune complexes with the bound antibody. The immune complex is incubated with an enzyme-linked secondary antibody which recognizes the same or a different epitope on the antigen as the bound antibody. Hence, enzyme activity is directly proportional to the amount of antigen in the biological sample. See Kemeny, D M, and S. J. Challacombe (eds), ELISA and Other Solid Phase Immunoassays, John Wiley & Sons, Chichester, 1988 which is incorporated by reference.

Typical enzymes that can be linked to secondary antibodies include horseradish peroxidase, glucose oxidase, glucose-6-phosphate dehydrogenase, alkaline phosphates, (β-D-galactosidase and urease. Secondary antigen-specific antibodies linked to various enzymes are commercially available from, for example, Sigma Chemical Company, St Louis, Mo. and Amersham Life Sciences, Arlington Height, Ill.

Competitive ELISAs are similar to noncompetitive ELISAs except that enzyme-linked antibodies compete with unlabeled antibodies in the blood sample for limited antigen binding sites. Briefly, a limiting number of antigens are bound to the reaction cell. Biological sample and enzyme labeled antibodies are added to the reaction cell. Antigen-specific antibodies in the biological sample compete with enzyme labeled antibodies for the limited number of antigens bound to the reaction cell. After immune complexes have formed, nonspecific binding is removed, enzyme substrate is added and the enzyme activity is measured. No secondary antibody is required. Because the assay is competitive, enzyme activity is inversely proportional to the amount of antibodies in the biological sample.

Homologous immunoassays can also be used when practicing the method of one possible embodiment of the present invention. Homogenous immunoassays may be preferred for low molecular weight analytes, such as hormones, therapeutic drugs, and illegal contraband that cannot be analyzed by other methods, or analytes found in high concentration. Homogeneous assays are particularly useful because no separation step is necessary. See Boguslaski, R. C., E. T. Maggio, R. M. Nakamura (eds), Clinical Immunochemistry: Principles of Methods and Applications, Little Brown, Boston (1984) hereby incorporated by reference.

In homologous techniques, bound or unbound antigens are enzyme-linked. When antibodies in the biological sample bind to the enzyme-linked antigen, stearic hindrances inactivate the enzyme. This results in a measurable loss in enzyme activity. Free antigens (i.e., not enzyme-linked) compete with enzyme-linked antigen for limited antibody binding sites. Thus, enzyme activity is directly proportional to the concentration of antigen in the biological sample.

Enzymes useful in homogeneous immunoassays include lysozyme, neuramidase, trypsin, papain, brorelain, glucose-6-phosphate dehydrogenase and (β-D-galactosidase. See Persoon, T., Immunochemical Assays in the Clinical Laboratory. Clinical Laboratory Science 5:31 (1992) hereby incorporated by reference. Enzyme-linked antigens are commercially available or can be linked using various chemicals well known in the art including glutaraldehyde and maleimide derivatives.

Fluorescent immunoassays can also be employed when practicing the method of one possible embodiment of the present invention. Fluorescent immunoassays are similar to ELISAs except that the enzyme is substituted for fluorescent compounds called fluorophores or fluorochromes. These compounds have the ability to absorb energy from incident light and reemit the energy as light of a longer wavelength and lower energy. Fluorescein and rhodamine, usually in the form of isothiocyanates which can be readily coupled to reactants and antibodies are most commonly used in the art. See Stites, D. P. et al., Basic and Clinical Immunology; Appleton & Lange, east Norwalk, Conn. (1994) hereby incorporated by reference. Fluorescein absorbs light of 490 to 495 nm in wavelength and emits green light at 520 nm in length. Tetramethylrhodamine absorbs light of 550 nm in wavelength and emits red light at 580 run in length.

Phycobiliproteins isolated from algae, porphyrins, and chlorophylls which all fluoresce at approximately 600 nm are also being used in the art. See Hemmila, I., Fluoroimmunoassays and Immunofluorometric Assays. Clin Chem, 31: 359 (1985) and U.S. Pat. No. 4,542,104 to Stryer et al. hereby incorporated by reference. Phycobiliproteins and derivative are commercially available under the names R-phycoerythrin (PE) and Quantum Red™ from for example, Sigma Chemical Company, St. Louis, Mo.

In addition, Cy-conjugated secondary antibodies and reactants are useful in immunoassays and are commercially available. Cy-3, for example, is maximally excited at 554 run and emits light of between 568 and 574 rim. Cy-3 is more hydrophilic than other fluorophores and thus has less of a tendency to bind nonspecifically or aggregate. Cy-conjugated include Cy-2, Cy-3, and Cy-5 are commercially available from Amersham Life Sciences, Arlington Height, Ill.

Chemiluminescence, electroluminescence and electrochemiluminescence (ECL) detection methods are also attractive means for quantifying analytes in a biological sample. Luminescence compounds have the ability to absorb energy which is released in the form of visible light upon excitation. In chemiluminescence, the excitation source is a chemical reaction; in electroluminescence the excitation source is an electric field; and in ECL an electric field induces a luminescent chemical reaction.

Molecules used with ECL detection methods generally comprise an organic ligand and a transition metal. The organic ligand forms a chelate with one or more transition metal atoms forming an organometallic complex. Various organometallic and transition metal-organic ligand complexes have been used as ECL labels for detecting and quantifying analytes in biological samples. Due to their thermal, chemical and photochemical stability, their intense emissions and long emission lifetimes, ruthenium, ossium, rhenium, iridium and rhodium transition metals are favored in the art. The types of organic ligands are numerous and include anthracene and polypyridyl molecules and heterocyclic organic compounds. For example, bipyridyl, bipyrazyl, terpyridyl, and phenanthrolyl, and derivatives thereof, are common organic ligands in the art. A common organometallic complex used in the art includes tris-bipyridineare ruthenium (II), commercially available from IGEN, Inc., Rockville, Md. and Sigma Chemical Company, St. Louis, Mo.

Advantageously, ECL can be performed under aqueous conditions and under physiological pH thus minimizing biological sample handling. See Leland, J. K. et al., “Electrogenerated Chemiluminescence: An Oxidative-Reduction Type ECL Reactions Sequence using Triprophyl Amine”, Journal of the Electrochemical Society (1990), vol. 137 #10, pp. 3127-3131, WO 90/05296, and U.S. Pat. No. 5541113 to Siddigi et al. hereby incorporated by reference. Moreover, the luminescence of these compounds may be enhanced by the addition of various cofactors such as amines.

In practice, a tris-bipyridineare ruthenium (II) complex, for example, may be attached to a secondary antibody using strategies well known in the art including attachment to lysine amino groups, cysteine sulfhydryl groups, and histidine imidazole groups. In a typical ELISA immunoassay, secondary antibodies would recognize IS antibody bound to antigens but not unbound antigens. After washing nonspecific binding complexes, the tris-bipyridine ruthenium (II) complex would be excited by chemical, photochemical and electrochemical excitation means, such as by applying current to the reaction vessel. See, e.g., WO 86/02734 to Bard, A. J. and Whiteside, G. M., which is herein incorporated by reference. The excitation would result in a double oxidation reaction of the tris-bipyridineare ruthenium (II) complex resulting in luminescence which could be detected by, for example, a photomultiplier tube. Instruments for detecting luminescence are well known and commercially available, for example from IGEN, Inc., Rockville, Md.

The techniques described above for detecting analytes in a biological sample are only exemplary of the many techniques that could be employed with one possible embodiment of the present invention. One skilled in the art will appreciate that one possible embodiment of the present invention can be modified to accommodate many other techniques including radioimmune assays (RIA), biotin-antibody conjugated assays, time resolved fluorescence, colloidal gold conjugates assays, ferritin conjugates assays, western blotting, variable number of tandem repeats assays, short tandem repeat assays and sex specific assays using probes for detecting human Y-specific regions.

Digitizing the Results

Once interactions between the reactants and analytes have been identified and quantified, the signals may be digitized for storage and to facilitate analysis. Regardless of whether a fluorescent dye (quantified using a fluorescence scanner) or other colorometric signal, e.g., Alkaline Phophatase—BCIP/NBT developing reagent (quantified using a conventional flatbed scanner), the spot density can be digitized and analyzed using any of a variety of commercially available imaging and densitometry software, such as for example, Adobe PHOTOSHOP®, Array Vision, Spotware, etc. An antibody fingerprint or conventional bar code on the slide preferably identifies the source of the biological sample used to generate the digital profile.

It will be appreciated by one skilled in the art that other methods of developing, imaging, storing and accessing the microarray spot densities may be employed.

### EXAMPLE 1

General Protocol Using Z-GRIP™

Bonding of PVDF to substrate—a) apply silicon, glue or double sided tape to solid substrate in even thin layer, b) under clean conditions, place sheet on lab bench and apply solid substrate (glue side facing PVDF sheet) to vinyl fluoride sheet, and c) press firmly and allow drying. Using an sharp instrument, e.g., a razor blade, exacto knife, etc., cut sheet so that it is size of solid substrate.

Specialized printing buffer—KPO₄ buffer (maintain desired pH), NaCl, and DTT. While some printing will occur if one of these reagents is not included, we have found that optimal printing consistency and immunochemistry occur with the following buffer: 20 mM KPO₄ (pH 7.4), 100 mM NaCl, and 0.01% DTT. After printing is complete, arrays can be used for protein-protein interaction studies or immunochemistry applications.

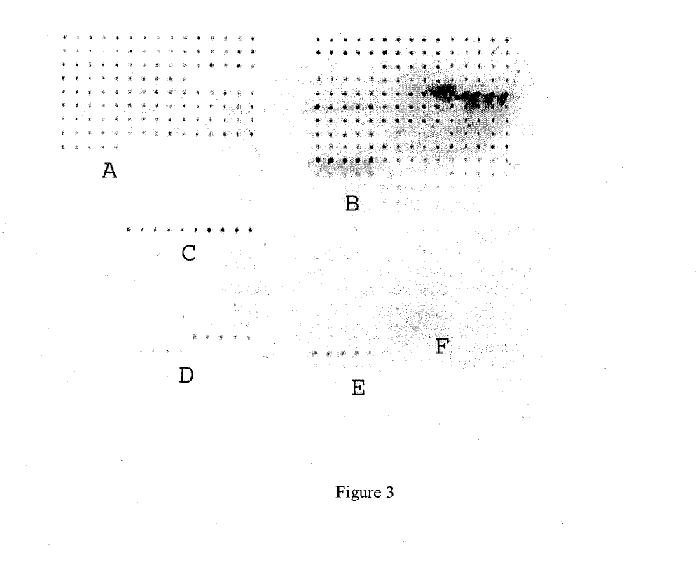

Immunochemistry applications—After printing, the Z-GRIP™ slide is blocked with a protein blocker, e.g., 2% Caseine was used in this case. As shown in FIG. 3A, the most consistent printing was observed in KPO₄ buffer, with NaCl, and DTT. FIG. 3B shows the membrane loading characteristics of PVDF under the conditions used. FIGS. 3C, 3D, 3E, and 3F show immunochemical interactions.

FIG. 3 shows an example of the substrate and immunochemistry. A and B sub-arrays allowed measurement of printing consistency and detection limits (Note some asymmetry was engineered into the array as a reference tool and that portions of B are beyond and below detection limits). C, D, E and F show the response of this Lupus patient to different disease markers used in the array. Non-autoimmune disease patients show no response to these disease markers (as in F). Also note that the actual size of each sub array is less than 0.5 cm.

### EXAMPLE 2

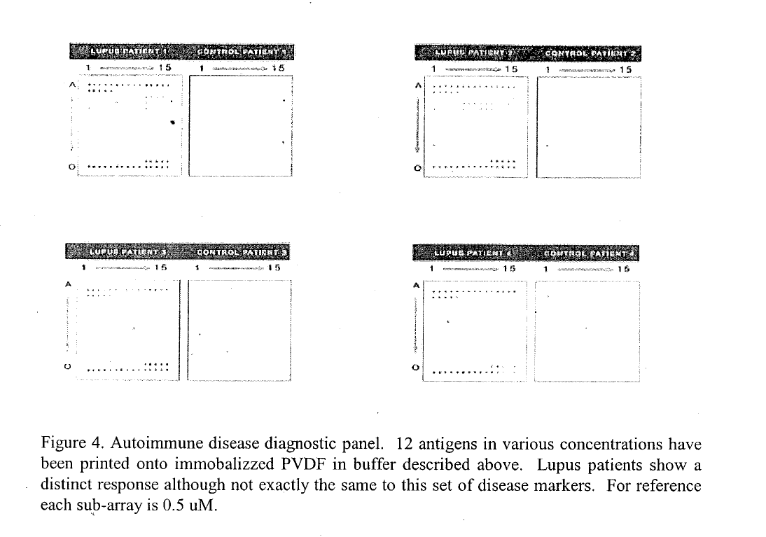

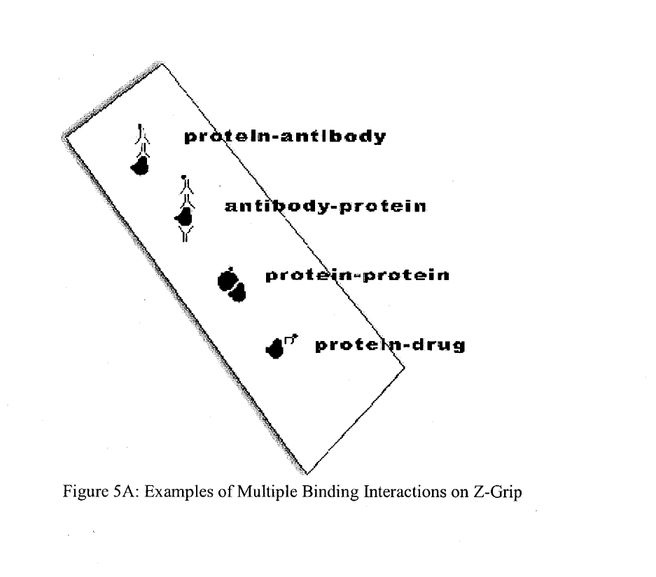

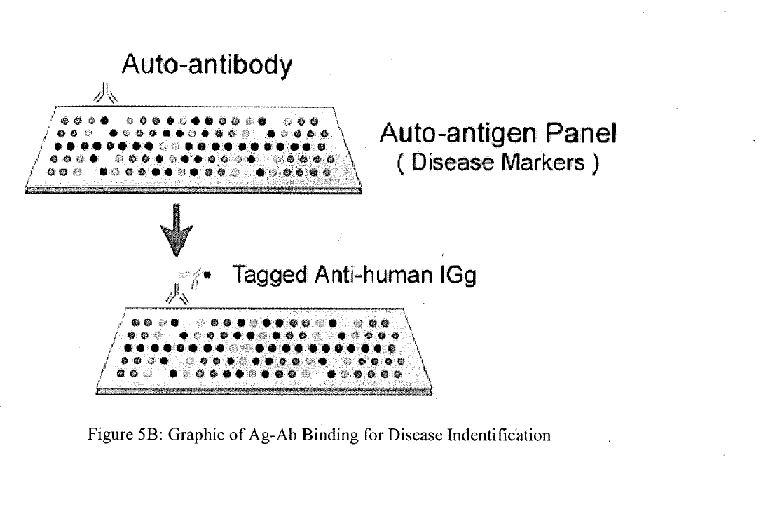

The surface was also used to determine differences in immunoreactivity to autoimmune disease related markers between 4 Lupus patients and 4 age/sex matched controls. Antigens were printed in 8 replicate arrays on substrate at a concentration of 1 mg/ml in optimized buffer described above on PVDF prepared as described above. The array was blocked with Casein and Patient serum was diluted to a titer of 1000 and a incubated with arrays for 1 hr. The arrays where then washed 3× in PBS and a secondary anti-human IgG conjugated to Alkaline Phosphatase was added (Pierce Biochemicals, Rockford Ill., Goat anti human IgG Alkaline Phosphatase Conjugated Product #31310) After 1 hr the arrays were washed 3× in PBS and a developing reagent was added (1 ml×BCIP/NBT, Pierce Biochemicals). After 15 minutes slides were washed, allowed to dry and scanned in a commercial scanner. Results are shown in FIG. 4. Although Alkaline Phosphatase conjugated secondary antibody was used, this method is compatible with protein A conjugated Alkaline phosphatase or secondary antibodies labeled with other enzymes (HRP) or dyes (fluorescent etc). FIG. 4 shows the background and specificity of this substrate in this use and utility for immunochemistry applications.

FIG. 4 shows the autoimmune disease diagnostic panel. 12 antigens in various concentrations were printed onto immobilized PVDF in buffer described above. Lupus patients show a distinct response although not exactly the same to this set of disease markers. For reference each sub-array is 0.5 uM.

Protocol for Detection of Antibodies to Target Proteins on Z-GRIP™ Substrate

All arrays are labeled and individually placed into petri-dishes, filled with 10 ml of Casein Blocker in PBS (Fisher Scientific) incubated at room temperature for 1 hour with shaking. Samples are diluted to various concentrations then added to the block solution with the arrays and allowed to incubate for 1 hour at room temperature with shaking. The Blocker PBS/samples/control solutions are discarded and arrays are then washed with 10 ml of 10× PBS wash buffer for 10 minutes with shaking. This last step is repeated three times and the wash solution discarded. After last wash add 10 ml of BCIP/NTB detector (Fisher Scientific) and allow development for 15 minutes with shaking. The developer is discarded and the reaction is stopped with dH2O for 2 minutes and the arrays allowed to dry for several hours or overnight.

### EXAMPLE 3

The Z-GRIP™ Substrate and protocol was used to observe the stability of an antigen over a period of two months. First the arrays were printed with antigens at the same locations, then stored at room temperatures for different periods of time, and then developed individually at discrete intervals. All the conditions kept constant, including the developing time and the amount of primary and secondary antibodies added. In all the slides, 10 ul of the primary and 1 ul of the secondary was added. Using the Z-GRIP™ protocol, each disease was repeated 5 times.

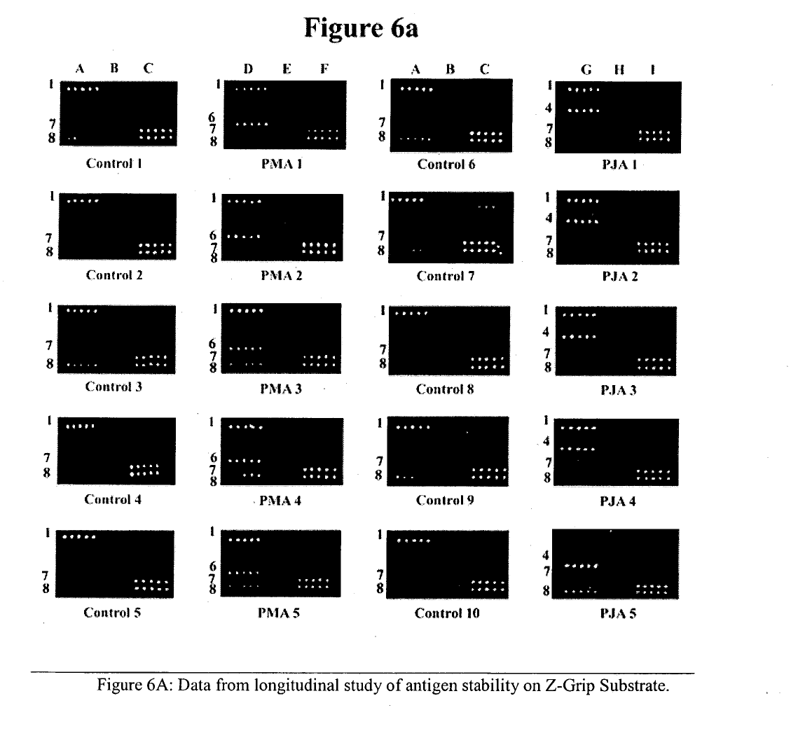

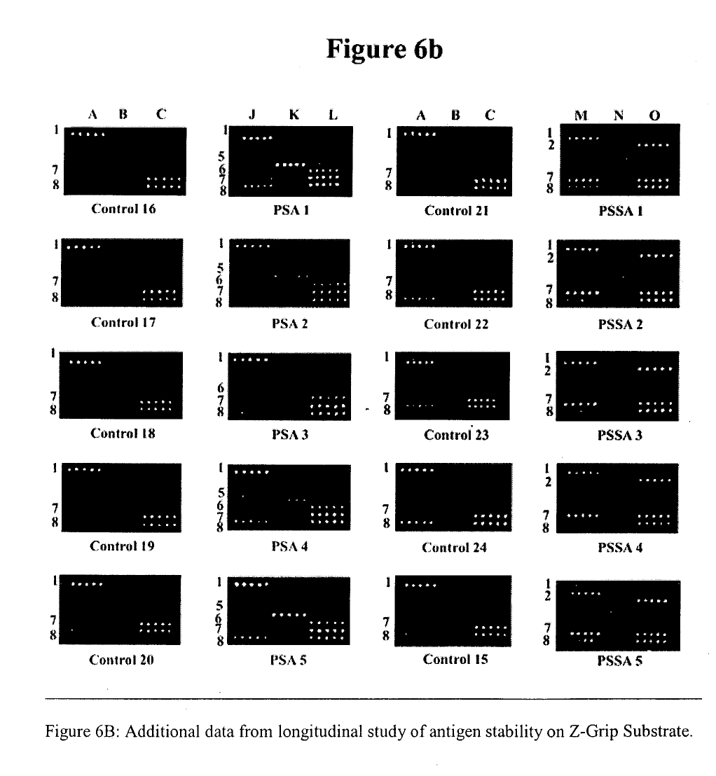

FIG. 6A shows stability results, illustrating the same set of antigens printed on 80× Z-GRIP™ Substrate over a period of two months. All antigens and human serums were freeze/thawed for each run, and tests were repeated five times, and positive responses were traced back to the antigens in the 384-wells dish as shown in Table 1. Four human serums were used, including patients having antibodies against Mitochondrial antigens (MIT), Histidyl tRNA Synthetase (Jo-1), Smith (Sm), or Sjogren Syndrome type B antigen (SSB). Control slides 1 to 10 are the normal human serum that acts as a negative control for the experiment. As expected, the three positive control location appears in both patient serum and normal human serum. The positive response at A6 for the positive patient for the Mitochondrial Antigen (PMA) and at A4 for the positive patient for Histidyl tRNA Synthetase (PJA), indicate the stability and reliability of the antigen and antibody. The sporadic appearance of dots at location A8 indicates a suspected contamination, which was solved by increasing the percentage of ethanol in the wash buffer, and cleaning the robotic printer.

FIG. 6B shows continuation of the experiment described in FIG. 6a. Patient positive for Smith antigens (PSA) have five dots at C6 (the Smith (Sm) antigen). There is also positive response at location B5 (the RNP antigen). The patient positive for Sjogren Syndrome type B antigens (PSSA), this responds to both C2 (Sjogren Syndrome type B antigen (SSB) from BioDesign) and A7 (SSB from Immunovision). Antigen locations are shown in Table 1.

TABLE 1
Antigens printed for Figures 6a and 6b.

| | A | B | C |
|---|---|---|---|
| 1 | Control+ve | | |
| 2 | | | SSB(BioD) |
| 3 | SmA(BioD) | pANCA(BioD) | cANCA(BioD) |
| 4 | Jo-1(BioD) | ApoH(BioD) | ScI-70(BioD) |
| 5 | Jo-1(immuno) | RNP(immuno) | ScI-70(immuno) |
| 6 | MIT(immuno) | DNA(immuno) | SmA(immuno) |
| 7 | SSB(immuno) | SSA(immuno) | Control+ve |
| 8 | IgA | | Control+ve |

Table 1. Diagram illustrating the location and type of antigens spotted on the Z-GRIP™ substrates featured in FIGS. 6a and 6b. “Control +ve” are positive controls used for calibration. Antigens purchased from BioDesign are labeled with (BioD), where antigens purchased from Immunovision are labeled with (immuno). Each box represents five dots of the same antigen (indicated above) spotted onto the Z-GRIP™ substrates using the robotic printer.

### EXAMPLE 4

The Z-GRIP™ Substrate and protocol was used to determine the sensitivity of the Z-GRIP™ assay for two different autoimmune diseases and show that different diseases can have overlapping markers. Titers of 1:500, 1:200 and 1:100 were used for the primary with secondary consisting of 1 ul in 10 ml of PBS. Antigen locations are shown in Table 2.

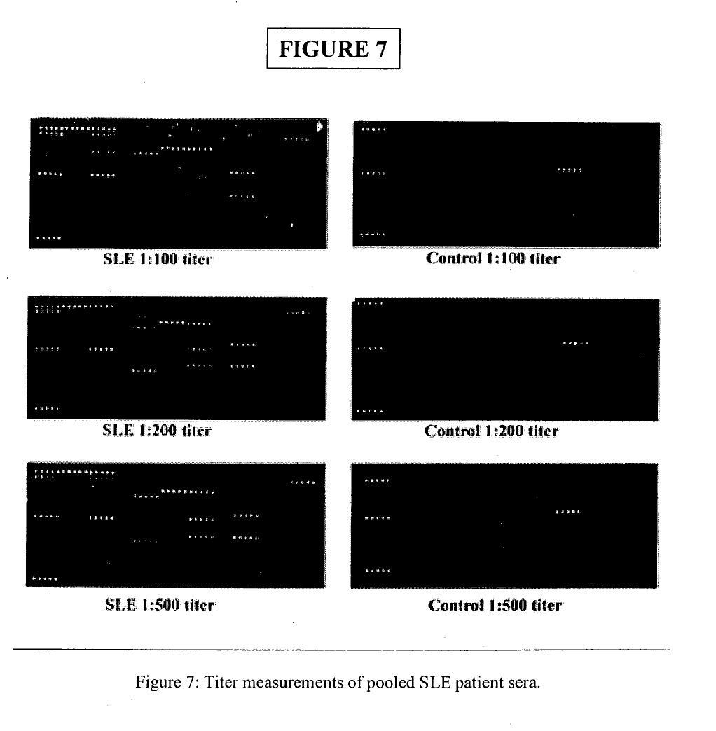

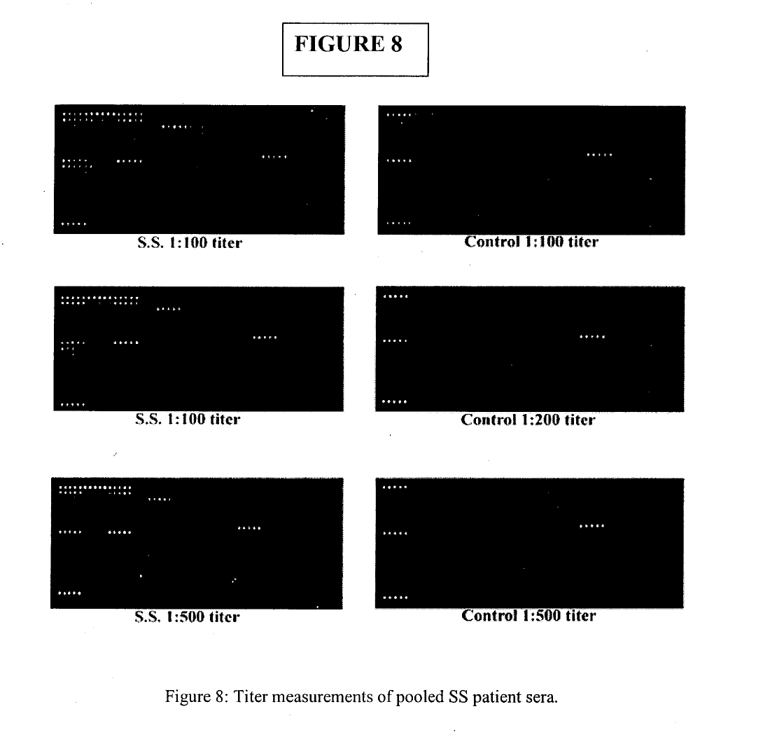

TABLE 2
Antigens printed for FIGS. 7 and 8

| | A | B | C | | E | F | G | H | I |
|---|---|---|---|---|---|---|---|---|---|
| 1 | Control +ve | SSA | SSA10/30 | SCL5/35 | SCL1/39 | | PCA1/39 | F2A2 his | F2A210/30 |
| 2 | SSA5/35 | SSA1/39 | SSB | Jo-110/30 | Jo-15/35 | Jo-11/39 | F2A25/35 | F2A21/39 | whole his |
| 3 | SSB10/30 | SSB5/35 | SSB1/39 | MIT | MIT10/30 | MIT5/35 | whole10/30 | whole5/35 | whole1/39 |
| 4 | | SmA | SmA10/30 | MIT1/39 | RAG | RAG10/30 | | | |
| 5 | SmA5/35 | SmA1/39 | SRC | RAG5/35 | RAG1/39 | Clq | H2b | H2b10/30 | H2b5/35 |
| 6 | SRC10/30 | SRC5/35 | SRC1/39 | Clq10/30 | Clq5/35 | Clq1/39 | H2b1/39 | H3&H4 | H3&H410/30 |
| 7 | | SCL | SCL10/30 | PCA | PCA10/30 | PCA5/35 | H3&H45/35 | H3&H41/39 | F4 |
| 8 | SCL5/35 | SCL1/39 | Jo-1 | PCA1/39 | F2A2 | F2A210/30 | F410/30 | F45/35 | F41/39 |
| 9 | Control +ve | | SSA10/30 | F2A25/35 | F2A21/39 | Whole his | Control +ve | | |
| 10 | SSA5/35 | SSA1/39 | | Jo-110/30 | Jo-15/35 | Jo-11/39 | whole10/30 | whole5/35 | whole1/39 |
| 11 | SSB10/30 | SSB5/35 | SSB1/39 | | MIT10/30 | MIT5/35 | H2b histone | H2b10/30 | H2b5/35 |
| 12 | | | SmA10/30 | MIT1/39 | | RAG10/30 | H2b1/39 | H3&H4 | H3&H410/30 |
| 13 | SmA5/35 | SmA1/39 | | RAG5/35 | RAG1/39 | | H3&H410/30 | H3&H41/39 | F4 |
| 14 | SRC10/30 | SRC5/35 | SRC1/39 | Clq10/30 | Clq5/35 | Clq1/39 | F410/30 | F45/35 | F41/39 |
| 15 | | | SCL10/30 | | PCA10/30 | PCA5/35 | | | |
| 16 | | | | | | | | | |
| 17 | | | | | | | | | |
| 18 | | | | | | | | | |
| 19 | | | | | | | | | |
| 20 | Control +ve | | | | | | | | |
| | A | B | C | D | E | F | G | H | I |

Table 2. Illustrating the location and type of antigens spotted on the Z-GRIP™ Substrates featured in FIGS. 7 and 8. “Control +ve” are positive controls used for calibration. The dilutions of the antigens are represented by numbers, where 10/30 indicates 10 μl of the original antigen in 30 μl of PBS, 5/35 indicates 5 μl of the original antigen in 35 μl of PBS, and 1/39 indicates 1 μl of antigen in 39 μl of PBS. Bolded antigens indicate positive responses to disease serum used in the experiments mentioned below. To be considered a positive response, each box/location will contain five purple spots on the Z-GRIP™ Substrate. Non-bolded antigens were spotted on the Z-GRIP™ Substrates, but did not react with the control or disease serum (as indicated by no spots at their location). All antigens were spotted using a robotic printer.

FIG. 7 shows the results of six arrays using a pool of five Systemic Lupus Erythematosus (SLE) patients' serum, or a pool of five, corresponding age and sex matched control patients' serum, for a range of titers. Positive responses can be traced back to the original antigen in the 384-wells dish as shown in Table 2. Positive control (Control +ve) for calibration is located at A1, A9, A20, and G9, and is used as a guide when the developing process is complete. All other antigens were diluted as pure, 10/30 (having a dilution of 10 μl original antigen in 30 μl Phosphate-buffered Saline (PBS)), 5/35 (having a dilution of 5 μl original antigen in 35 μl PBS), and 1/39 (having a dilution of 1 μl original antigen in 39 μl PBS). Pure Sjogren Syndrome type A antigen (SSA) is positive at B1, where SSA 10/30 is positive at C1 and C9, and SSA 5/35 is positive at A2. Pure Sjogren Syndrome type B antigen (SSB) is positive at C2. The Smith/RNP complex antigen (SRC) is positive at C5, and the Mitochondial antigen (MIT) is positive at D3. As for the Recombination Activating protein (RAG), Pure RAG is positive at E4, RAG 10/30 is positive at F4 and F12, and RAG 5/35 is positive at D5 and D13. Autoantibodies against histones are also present in SLE patients. The positive histones are Whole histones at F9 and I2, F2A2 at H1, pure H2b at G12, H2b 10/30 at H12, and H2b 5/35 at I12. On the corresponding control slide, only Control +ve react.

Results of six arrays using a pool of five Sjogren Syndrome (SS) patients' serum, or a pool of five, corresponding age and sex matched control patients' serum, for a range of titers is shown in FIG. 8. Positive responses can be traced back to the original antigen in the 384-wells dish, as shown in Table 2. Positive control (Control +ve) for calibration is located at A1, A9, A20, and G9, and is used as a guide when the developing process is complete. All other antigens were diluted as pure, 10/50 (having a dilution of 10 μl original antigen in 30 μl Phosphate-buffered Saline (PBS)), 5/35 (having a dilution of 5 μl original antigen in 50 μl PBS), and 1/50 (having a dilution of 1 μl original antigen in 39 μl PBS). Antigens that respond to the SS patient serum include Pure Sjogren Syndrome type A antigen (SSA) at B1, where SSA 10/30 is positive at C1 and C9, and SSA 5/35 is positive at A2 and A10. Pure Sjogren Syndrome type B antigen (SSB) and the Mitochondrial antigen (MIT) also respond to SS and are seen at C2 and D3 (respectively).

### EXAMPLE 5

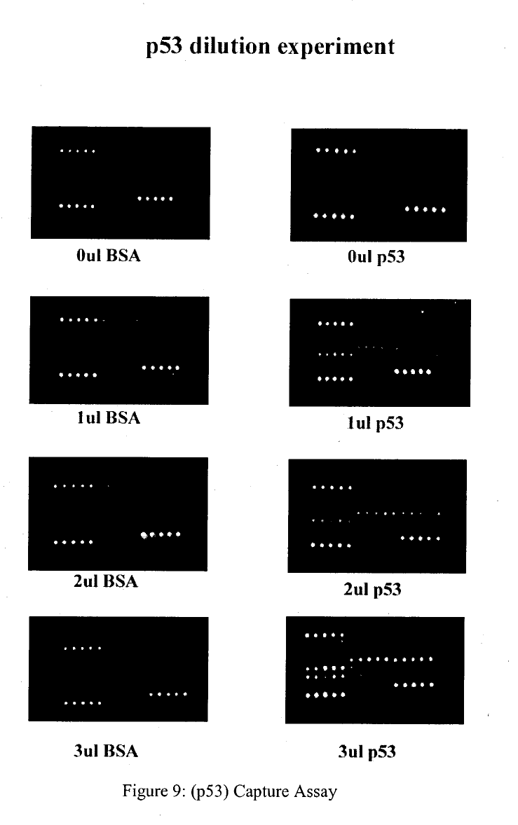

The Z-GRIP™ Substrate and modified protocol was used to demonstrate the utility of the Z-GRIP™ Substrate for a wide variety of applications such as for a (p53) capture assay (FIG. 9). It has previously been proven that antigens and different diseases have specific markers and can be captured on the Z-GRIP™ Substrate. Usually, the sandwich of the assay starts from the primary antigens that binds to the Z-GRIP™, and the next layer is the primary antibodies from a patient serum, and the next layer is the secondary antibodies that recognize the primary antibodies and bind to them. However, in the p53 sandwich, the first layer that is attached to the substrate is an antibody (which is the mouse monoclonal p53 antibody). We then added the pure protein antigen (p53). The third layer of the assay is the primary antibodies (rabbit anti p53), and the fourth layer is the secondary antibodies (anti-rabbit AP) for detection.

The volume of the pure protein p53 varies, and the BSA solution is the negative control for this experiment. We used 0, 1, 2, and 3 μl aliquots for both the BSA and p53. The rabbit anti p53 that was used is 40 μl and the secondary with the AP is 10 μl for each slide.

The protocol for this experiment is the same as Z-GRIP™ protocol, except for the following additional steps. After shaking substrate/blocker at room temperature for 1 hour titers of 0, 1, and 2 μl of BSA and p53 pure protein solution are added to each petri-dish, then after the three wash cycles 40 μl of rabbit anti-p53 are added to the petri-dish and shaken for 1 hour at room temperature. The solutions are then discarded and 10 ml of PBS is added and mixed for 10 minutes, and repeated three times. After the last wash is discarded 10 ml of PBS with 10 μl of anti-rabbit AP is added to each petri-dish/substrate/wash solution combination and incubated for 1 hour, followed by three more washes. Results are shown in FIG. 9.

### EXAMPLE 6

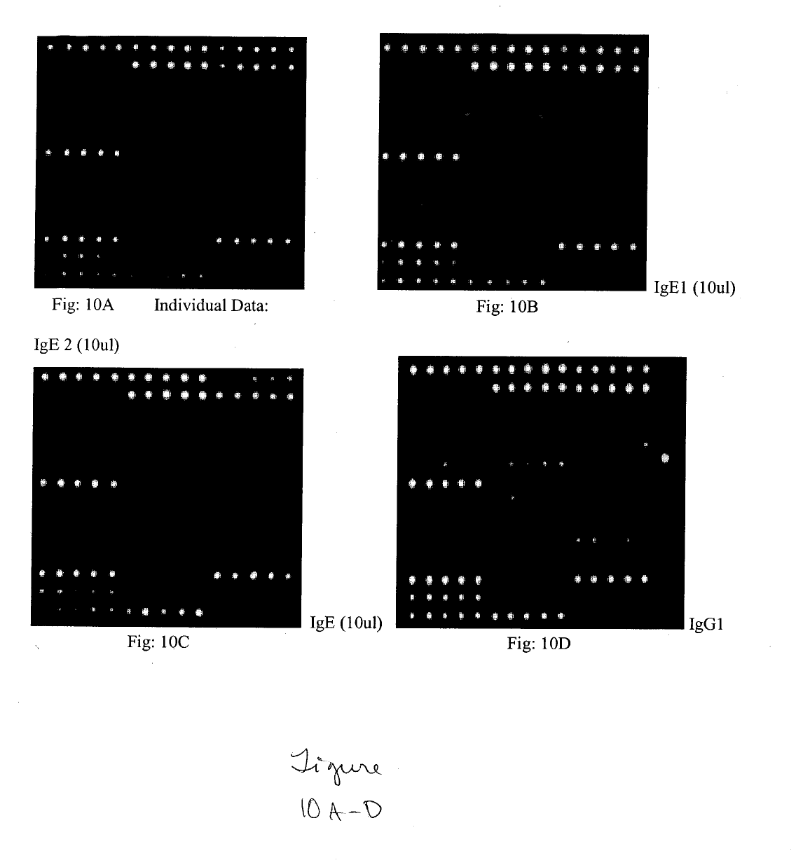

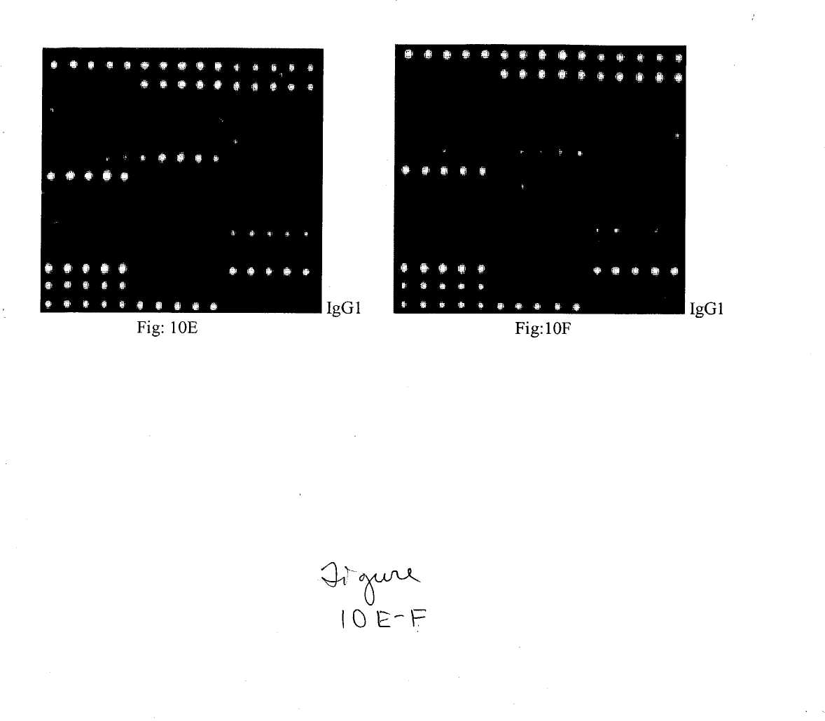

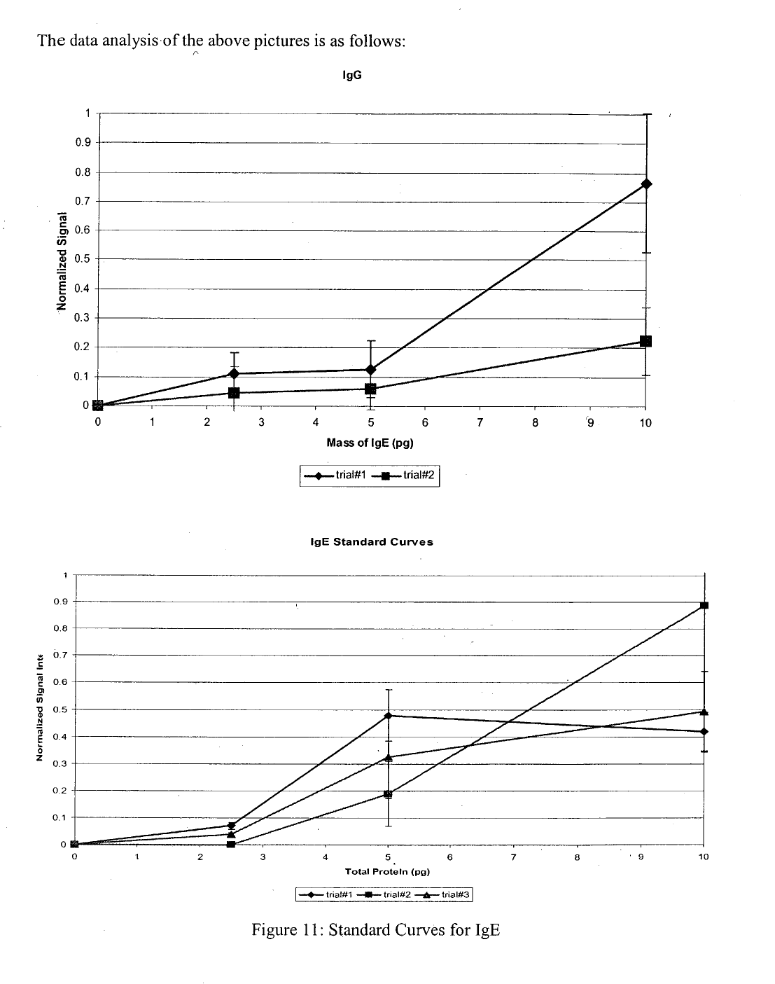

The Z-GRIP™ Substrate and protocol was used to determine if the assay was capable of detecting low concentrations of immunoglobulins such as IgE (see FIG. 10) compared to IgG, which is normally present in higher concentrations. Antigens (including the pure protein IgE and IgG1) and antibodies against the two different antigens were spotted onto the substrate. The two standard pure proteins acts as a standard curve and could estimate the minimum and maximum titer range in which the future experiments can be based on. The experiment also includes quantitative results sections for plotting a standard curve (see FIG. 11) both for the IgG1 and the three 10 μl of the IgE secondary antibody, and the IgE. The array includes three controls, three 10 μl of the IgG1 secondary antibody and also three 10 μl of the IgE secondary antibody. Well locations are shown in Table 3.

TABLE 3
Well location table:

| | | | | | |
|---|---|---|---|---|---|
| A1 | anti-IgG-AP 1:10 | A2 | anti-IgG-AP 1:10 | A3 | anti-IgG-AP 1:100 |
| A4 | | A5 | anti-IgG₁-AP 1:50 | A6 | anti-IgG₁-AP 1:100 |
| A7 | IgG₁ 1:100T | A8 | IgG₁ 1:20T | A9 | IgG1₁ 1:10T |
| A10 | IgG₁ 1:5T | A11 | IgG₁ 1:2T | A12 | IgG₁ 1:1T |
| A13 | IgG₁ 1:800 | A14 | IgG₁ 1:500 | A15 | IgG₁ 1:200 |
| A16 | IgG₁ 1:100 | A17 | IgG₁ 1:50 | A18 | |
| A19 | anti-IgG-AP 1:10 | A20 | anti-IgG-AP 1:100 | A21 | |
| A22 | IgE 1:100T | A23 | IgE 1:20T | A24 | IgE 1:10T |
| B1 | IgE 1:5T | B2 | IgE 1:2T | B3 | IgE 1:1T |
| B4 | IgE 1:800 | B5 | IgE 1:500 | B6 | IgE 1:200 |
| B7 | | B8 | | B9 | |
| B10 | anti-IgG-AP 1:10 | B11 | | B12 | anti-IgE-AP 1:50 |
| B13 | anti-IgE-AP 1:100 | B14 | | B15 | |
| B16 | IgE 1:100 | B17 | IgE 1:50 | B18 | |
| B19 | | B20 | | B21 | |

Table 3. Location and type of antigens spotted on the Z-GRIP™ Substrates

Antigen Preparation

Approximately 1 liter suspension HeLa Cells (ATCC Type S3) were grown to mid log phase (6×10⁵ cells/ml). Cells were grown in Dulbbecco's Modified Eagles Medium plus 1000 mg glucose/L and L-glutamine (Fisher Chemical) plus 5% Newborn Calf Serum using a 37° C. spin culture apparatus (Fisher Chemical). Cells were collected by centrifugation (20 min 5 K, Sorval ss34) and washed three times in Phosphate Buffered Saline (PBS). 50 ul of ice-cold PBS with broad-spectrum protease inhibitor cocktail (Sigma Chemical) was added to the cell pellet and placed on ice (all subsequent manipulations done on ice). The pellet was suspended by gentle tapping on test tube (approximately 10 times). The suspended cell pellet was sonicated with a sonicator setting of 6 using the large tip. Sonication was for 10 rounds of 10 sec with each round being followed by a 10 sec cooling on ice. Cell disruption was verified using a light microscope. Cell debris was removed by centrifugation (20 min, 9K, Sorval ss34). Supernatant was placed in boiled dialysis membrane (3-5 kd cut-off) and dialyzed 2-times against 2 liters Buffer A (20 mM KPO4 pH 7.4, 20 mM KCl) for 11 hours and then 2 hours respectively. Lysate was then placed in an ice-chilled beaker with magnetic stir bar on ice on stir plate set at lowest setting that allowed mixing. Hand ground ammonium sulfate (AS, Fisher chemical) was added several grains at a time over the course of 1 hour to a final AS concentration of 30%. The precipitate was collected by centrifugation (20 min, 9K, Sorval ss34) and the supernatant was added to a second ice-chilled beaker as described above. Ground AS was added several grains at a time to a final AS concentration of 90%. The pellet was removed by centrifugation (20 min, 9K, Sorval ss34) and suspended in 10 ml of buffer A. The lysate was then placed in a boiled dialysis membrane (3-5 kd cut-off) and dialyzed 2 times against 2 Liters Buffer A for 11 hours and then 2 hours respectively. The lysate was then removed from dialysis bag and filtered through a 0.2 μM filter and stored on ice.

Chromatography

Four liters of Buffer A (20 mM KPO4 pH 7.4, 20 mM KCl) and one liter buffer B (20 mM KPO4 pH 7.4, 500 mM KCl) were autoclaved and chilled. Buffer used for chromatography were additionally filtered sterilized (0.2 uM, Fisher Chemical). The chromatography FPLC (Pharmacia) was washed and fitted with 10 ml loading chamber and 5 ml strong anion exchange column (HiTrap Q, Pharmacia). The column was prepared for lysate by the following protocol: 60 min buffer A, 20 min Buffer B, 20 min Buffer A, 20 min buffer B, 60 min buffer A at a flow rate of 1 ml/min. The lysate was added to the loading chamber and using a syringe. The FPLC was programmed to load sample and conduct a 50 minute buffer A wash (10 column volumes) followed by a ramp from 100% buffer A to 100% buffer B at a flow rate of ½ ml/minute. The collector was set to collect 90 drops. Chromatography revealed that protein eluted off the column starting at about 50 mM KCL. The elution profile was consistent with past elution profiles performed under similar conditions. The fractions were capped, numbered and frozen on crushed dry ice. Once frozen, fractions were transferred to −70° C.

Array Spotting

PVDF (0.45 um pore size, Millipore) was cut into 5 by 8 cm pieces and marked to indicate the locations where column fractions were to be spotted. Chromatography fractions were thawed at 37° C., placed on ice and 5 μl of each column fraction was applied using a micropipette. The arrays were then covered with a Plexiglas dust cover and allowed to dry overnight.

Serum Incubations

Normal human serum in transfusion bags (gift from the U.C. Irvine Medical Center) aliquoted into 50 and 10 ml sterile tubes and frozen at −70° C. Each serum sample was assigned a number that can be traced back to original patient information (sex, blood type, proof of negative HIV, HepA, Hep B). Four arrays were labeled and then washed 3 times in 20 ml PBS 30 min each. All washing and incubations occurred at room temperature (25° C.), with rotation in 12×15 ml plastic trays. 10 ml of a 2% nonfat milk solution in PBS was added and the membranes were blocked for 45 minutes. 330 μl of each serum sample from four individuals (from now on called individuals1−4° C.) was added to the appropriate nonfat milk/PBS solution and incubated for 60 min. This solution was then removed and the arrays were washed with 20 ml PBS 3 times for 20 min each. After washing the arrays were suspended in 10 ml of PBS and 1 μl of goat anti human IgG (fc region) monoclonal antibody linked to HRP (Pharmacia) was added to PBS and the solution was incubated for 60 min. This solution was then removed and the arrays were washed with 20 ml PBS 3 times for 20 min each. The arrays were placed on saran wrap and a chemiluminescent reagent (ECL, Pharmacia) was added. The membranes were wrapped in saran wrap, placed in a film cassette and used to expose X-ray film (Fuji Super RX). The resulting film was developed using a standard darkroom film processor and washed and dried. The film was scanned using a desktop scanner and a composite figure showing all four arrays was created in Adobe PHOTOSHOP®.

Results

All 4 arrays are identical with respect to spot fraction number, volume and geometry. The difference in spotting pattern is due to a difference in IgG antibodies that are present in different individual serums (individual specific antibodies). These results demonstrate that unique individual-specific antibody fingerprints can be generated by contacting patient serum samples with an antigen array comprising a panel of antigens at predetermined locations on the array.

What is claimed is:

1. A substrate for protein immobilization, comprising a rigid support and a hydrophobic polymeric layer attached to said rigid support, wherein said hydrophobic polymeric layer has a surface chemistry adapted to immobilize a protein sample and wherein said substrate is configured to allow immobilization of a plurality of protein samples on discrete addressable spots thereon.

2. The substrate of claim 1, wherein said hydrophobic polymeric layer is a PVDF layer.

3. The substrate of claim 2, wherein the thickness of the PVDF layer is about 100-250 μm.

4. The substrate of claim 2, wherein the PVDF layer has a pore size of 0.2 to 0.45 μm.

5. The substrate of claim 2, wherein the PVDF layer is a sheet, a membrane, or is formed from PVDF pellets.

6. The substrate of claim 1, wherein said hydrophobic polymeric layer produces a background signal upon protein imaging using visual or fluorescent light of less than about 100 lumens.

7. The substrate of claim 1, wherein said hydrophobic polymeric layer produces a background signal upon protein imaging using visual or fluorescent light of between about 0 and 50 lumens.

8. The substrate of claim 1, wherein said hydrophobic polymeric layer produces a background signal upon protein imaging using visual or fluorescent light of between about 0 and 15 lumens.

9. The substrate of claim 1, wherein the rigid support is a silanated material.

10. The substrate of claim 1, wherein the rigid support is glass or plastic.

11. The substrate of claim 1, further comprising a bar code.

12. The substrate of claim 1, further comprising a removable protective film for protecting the hydrophobic polymeric layer and any protein samples immobilized thereon.

13. The substrate of claim 1 which further comprises a template attached to the rigid support, wherein said template divides said hydrophobic polymeric layer into at least two distinct sections, each section being configured to allow immobilization of a plurality of protein samples, and wherein said template is adapted to allow application of different chemical reagents to the at least two distinct sections of the polymeric layer.

14. A method of preparing a protein array, comprising the step of spotting a plurality of protein samples at discrete, addressable locations onto the PVDF layer of the substrate of claim 2, wherein the PVDF layer is in a dry state.

15. A method of making a PVDF-coated rigid substrate adapted to immobilize a plurality of bioactive molecules, comprising:
applying an adhesive layer to a rigid support;
adhering a PVDF membrane sheet to said adhesive layer on said rigid support;
drying; and
cutting excess PVDF membrane sheet away from the rigid support, to form the PVDF-coated rigid substrate.

16. The method of claim 15, wherein the adhesive is selected from the group consisting of a double-sided inert adhesive microfilm, a silicone, a glue, a double-sided tape and direct chemical bonding.

17. The method of claim 15, wherein the rigid support is glass or plastic.

18. The method of claim 15, further comprising attaching a template to the PVDF-coated rigid substrate.

19. The method of claim 18, wherein the template provides from about 2-500 wells.

20. An array-based kit for screening a patient for a plurality of autoimmune diseases, said kit comprising a substrate having a layer of PVDF attached to a rigid support, said layer of PVDF having a plurality of protein antigens related to said autoimmune diseases immobilized thereon at different known locations; and a reagent for detecting the binding of an antibody from the patient to the protein antigens.
